(19) United States
(12) Patent Application Publication
(10) Pub. No.: US 2003/0077288 A1
(43) Pub. Date: Apr. 24, 2003

(54) Compositions and methods for treatment of muscle wasting

(76) Inventors: Alfred L. Goldberg, Chestnut Hill, MA (US); Marcelo D. Gomes, Brookline, MA (US); Stewart H. Lecker, Brookline, MA (US); R. Thomas Jagoe, Ellesmere (GB)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/050,686

(22) Filed: Jan. 16, 2002

Related U.S. Application Data
(60) Provisional application No. 60/262,090, filed on Jan. 16, 2001.

(51) Int. Cl. A61K 38/43; C07K 14/435

(52) U.S. Cl. 424/185.1; 424/94.1; 530/350

(57) ABSTRACT

The present invention relates to the isolation of cell- or tissue-specific F-box proteins which are involved in ubiquitin-mediated protein degradation in a specific cell- or tissue-type. Accordingly, the invention provides nucleic acids and the proteins encoded by said nucleic acids which play a role in the ubiquitinylation and subsequent degradation of substrate proteins and in regulating cell proliferation, cell differentiation, and cell survival. The invention also provides methods for modulating protein degradation, cell proliferation, cell differentiation and/or cell survival by modulating protein ubiquitination; assays for identifying compounds which modulate protein degradation, cell proliferation, differentiation and/or cell survival; methods for treating disorders associated with aberrant protein degradation, cell proliferation, cell differentiation, and/or cell survival; and diagnostic and prognostic assays for determining whether a subject is at risk of developing a disorder associated with an aberrant protein degradation, cell proliferation, cell differentiation, and/or survival.

### CROSS-REFERENCE TO RELATED APPLICATIONS

This application claims the benefit of U.S. Provisional Application No. 60/262,090, filed Jan. 16, 2001, the contents of which are specifically incorporated by reference herein.

### GOVERNMENT SUPPORT

This invention was made with government support under grant No. NCC9-58-1 by the National Space Biomedical Research Institute and grant No. K08-DK02707 from the National Institutes of Health. The government has certain rights in the invention.

### BACKGROUND OF THE INVENTION

The ubiquitin-mediated proteolysis system is the major pathway for the selective, controlled degradation of intracellular proteins in eukaryotic cells. Ubiquitin modification of a variety of protein targets within the cell appears to be important in a number of basic cellular functions such as regulation of gene expression, regulation of the cell-cycle, modification of cell surface receptors, biogenesis of ribosomes, and DNA repair. One major function of the ubiquitin-mediated system is to control the half-lives of cellular proteins. The half-life of different proteins can range from a few minutes to several days, and can vary considerably depending on the cell-type, nutritional and environmental conditions, as well as the stage of the cell-cycle.

Targeted proteins undergoing selective degradation, presumably through the actions of a ubiquitin-dependent proteosome, are covalently tagged with ubiquitin through the formation of an isopeptide bond between the C-terminal glycyl residue of ubiquitin and a specific lysyl residue in the substrate protein. This process is catalyzed by a ubiquitin-activating enzyme (E1) and a ubiquitin-conjugating enzyme (E2), and in some instances may also require auxiliary substrate recognition proteins (E3s). Following the linkage of the first ubiquitin chain, additional molecules of ubiquitin may be attached to lysine side chains of the previously conjugated moiety to form branched multi-ubiquitin chains.

The conjugation of ubiquitin to protein substrates is a multi-step process. Ubiquitin is a small, highly conserved protein, which must be activated before it is transferred to a substrate protein. Activation of ubiquitin occurs through formation of a thioester bond between the COOH terminus of the ubiquitin molecule and a ubiquitin-activating enzyme, E1. Ubiquitin is then transesterified to one member of a family of a ubiquitin conjugating enzymes, E2 enzymes. Ubiquitin is then transferred, either directly or indirectly, to a lysine residue of a substrate protein. Transfer to the substrate protein may require the assistance of a ubiquitin ligase also termed E3 enzyme or complex. An E3 is generally required for the formation of multiubiquitin chains on the substrate, a step that facilitates efficient recognition of the substrate by the proteosome. It has been suggested that E3 is the primary source of substrate specificity in the ubiquitination cascade, as some E3s have been shown to directly bind substrates (Hershko et al. (1986) J. Biol. Chem. 261:11992; Bartel et al. (1990) EMBO J. 9:3179). Furthermore, in some situations, a ubiquitin molecule is first transferred from an ubiquitin conjugating enzyme to an E3 enzyme or complex, prior to being transferred to the substrate protein (Willems et al., supra).

To date, four classes of E3 enzymes have been identified which target different types of substrates: (i) E3α which targets proteins for ubiquitin dependent degradation based on the N-terminal amino acid residue of the substrate polypeptide (Varshavsky, Trends Biochem. Sci 22: 383-387 (1997)); (ii) the HECT domain proteins, exemplified by E6-AP which is involved in regulating the degradation of the tumor suppressor protein p53 (Scheffner et al., Cell 75: 495-505 (1993)); (iii) the Anaphase Promoting Complex (APC or cyclosome) which is a multisubunit complex that targets mitotic regulatory proteins for destruction via a ‘destruction box’ motif (Townsley and Ruderman, Trends Cell Biol. 8: 238-244 (1998) and Amon et al., Cell 77: 1037-1050 (1994)); and (iv) the SCF complexes which are multisubunit complexes comprising Skp1, Cdc53 (or another cullins protein), an F-box protein, and Rbx1 and are involved in regulating the destruction of a variety of proteins including G1 cyclins and Cdk inhibitors (Patton et al., Trends Genet. 14: 236-243 (1998) and Craig and Meyers, Prog. Biophys. & Mol. Biol. 72: 299-328 (1999)). F-box proteins contain an ‘F-box motif’ which is believed to be involved in the protein-protein interaction of the F-box with Skp1 component of the SCF complex. F-box proteins are thought to recruit specific substrate proteins for ubiquitin mediated degradation through other protein-protein interactions domains, such as WD40 or leucine rich repeat motifs.

A decrease in muscle mass, known as muscle wasting or cachexia, has been shown to be associated with the ubiquitin-dependent proteolytic system. Rats bearing the Yoshida AH-130 ascites hepatoma for 7 days showed a significant decrease in muscle mass in relation to non-tumor bearing controls (Llovera M. et al. (1995) Int. J. Cancer 61: 138-141). The muscle wasting was found to be associated with an increased proteolytic rate related to the ubiquitin-dependent proteolytic system. Muscle wasting is common among human cancer patients. In addition to cancer, ubiquitin-dependent muscle wasting is also influenced by nutritional manipulation (such as fasting and dietary protein deficiency), muscle activity and disuse, AIDS, and the pathological conditions, sepsis, trauma, and acidosis (Attaix D. et al. (1994) Reprod. Nutr. Dev. 34: 583-597). In a rat model for long lasting sepsis, researchers found that E2 mRNA levels increase during the acute and chronic disease phases and parallel a rise in muscle protein breakdown (Voisin L. et al. (1996) J. Clin. Invest. 97: 1610-1617).

“Cachexia” is the name given to a generally weakened condition of the body or mind resulting from any debilitating chronic disease. The symptoms include severe weight loss, anorexia and anemia. Cachexia is normally associated with neoplasmic diseases, chronic infectious diseases or thyroiditis, and is a particular problem when associated with cancerous conditions.

Indeed, it has been reported that a large proportion of the deaths resulting from cancer are, in fact, associated with cachexia, as also are various other problems commonly experienced by cancer patients, such as respiratory insufficiency, cardiac failure, diseases of the digestive organs, hemorrhaging and systemic infection (U. Cocchi, Strahlentherapie, 69, 503-520 (1941); K. Utsumi et al., Jap. J. Cancer Clinics, 7, 271-283 (1961)).

Cancer associated cachexia, which decreases the tolerance of cancer patients to chemotherapy and radiotherapy is said to be one of the obstacles to effective cancer therapy (J. T. Dwyer, Cancer, 43, 2077-2086 (1979); S. S. Donaldson et al., Cancer, 43, 2036-2052 (1979)). In order to overcome these problems, it used to be common for cancer patients with cachexia to receive a high fat and high sugar diet, or they used to be given high calorie nutrition intravenously. However, it has been reported that symptoms of cachexia were rarely alleviated by these regimens (M. F. Brenann, Cancer Res., 37, 2359-2364 (1977); V. R. Young, Cancer Res., 37, 2336-2347 (1977)).

The ability to selectively modulate ubiquitin-mediated proteolysis may provide the means for treating diseases associated with protein degradation, such as muscle wasting syndrome. Current inhibitors of the ubiquitin-proteasome pathway (i.e. proteasome inhibitors) affect protein degradation in all tissues, and thus are potentially toxic. Identification of a component of the ubiquitin mediated degradation pathway which is expressed in a tissue- or disease-specific manner would be an ideal target for therapeutics.

### SUMMARY OF THE INVENTION

One aspect of the present invention relates to isolated and/or recombinant forms of a cell- or tissue-specific F-box proteins, and portions thereof. For instance, there is provided isolated and/or recombinant polypeptides having an amino acid sequence identical or homologous (e.g., at least 65, 75, 85 or 95%) to the amino acid sequence as set forth in FIG. 5B. The cell- or tissue-specific F-box polypeptide can have an amino acid sequence encoded by a nucleic acid which hybridizes under stringent conditions to the nucleotide sequence set forth in FIG. 5A.

In another embodiment, other isolated and/or recombinant cell- or tissue-specific F-box polypeptides are provided, e.g., having an amino acid sequence identical or homologous (e.g., at least 65, 75, 85 or 95%) to the amino acid sequence set forth in FIG. 5B. The cell- or tissue-specific F-box polypeptide can have an amino acid sequence encoded by a nucleic acid which hybridizes under stringent conditions to the nucleotide sequence set forth in FIG. 5A.

The cell- or tissue-specific F-box polypeptides of the present invention are preferably encoded by a vertebrate gene, more preferably a mammalian gene, and even more preferably a human gene.

In preferred embodiments, the cell- or tissue-specific F-box polypeptides can be used as components of a ubiquitin ligase complex, e.g., which catalyze ubiquitinylation of a substrate protein. For instance, the polypeptide is capable of interacting with at least one other protein selected from the group consisting of ubiquitin, a component of a ubiquitin ligase, a skp1 protein, an Rbx1 protein, a ubiquitin conjugating enzyme, a cullins, and a substrate protein.

Still another aspect of the present invention provides nucleic acids which encode the subject cell- or tissue-specific F-box polypeptides, e.g., which nucleic acid hybridize under stringent conditions to a nucleic acid probe having a nucleotide sequence represented by at least 20, 40, 60, 80 or 100 consecutive nucleotides of the sequence set forth in FIG. 5A, or a sequence complementary thereto. In a preferred embodiment, the nucleic acid comprises the nucleotide sequence set forth in FIG. 5A.

The subject nucleic acid can be used to generate expression constructs, such as by placing a transcriptional regulatory sequence in operable linkage with the cell- or tissue-specific F-box polypeptide coding sequence. Accordingly, expression vectors encoding the subject polypeptides can be generated using expression vectors capable of replicating in at least one of a prokaryotic cell and a eukaryotic cell. Preferred substrate polypeptides are regulatory components of muscle cells or components of the myofibrillar apparatus.

Thus, another aspect of the present invention pertains to a host cell transfected with such an expression vector, e.g., expressing recombinant cell- or tissue-specific F-box polypeptides, as well as methods of producing a recombinant cell- or tissue-specific F-box polypeptide by culturing the instant cell to express the recombinant polypeptide.

The present invention also relates to transgenic animals having cells which harbor a transgene encoding a recombinant cell- or tissue-specific F-box polypeptide, or in which the endogenous gene has been inactivated, e.g., by homologous recombination.

Still another embodiment of the present invention relates to an isolated nucleic acid which selectively hybridizes under high stringency conditions to at least ten nucleotides of a nucleic acid sequence represented by the sequence set forth in FIG. 5A, or complementary sequences thereof, which nucleic acid can specifically detect or amplify a nucleic acid sequence of a vertebrate cell- or tissue-specific F-box polypeptide. Such nucleic acid can be used, e.g., to generate the expression constructs described above, as well as various assays for detecting cell- or tissue-specific F-box genes or transcripts, or for antisense therapy. In a preferred embodiment, the nucleic acid is labeled.

Yet another aspect of the present invention provides reconstituted protein mixtures or cell lysates including a cell- or tissue-specific F-box polypeptide, along with a substrate protein. The mixture may further include ubiquitin, an E1 enzyme, an E2 enzyme, a cullins protein, a Skp1 protein and/or an Rbx1 protein. As appropriate, the E1, E2 or cell- or tissue-specific F-box enzymes used to charge the mixture can be provided as transiently ubiquitinated intermediates.

Still another aspect of the present invention pertains to an assay for identifying an inhibitor of cell- or tissue-specific F-box-mediated ubiquitination. For example, in one embodiment the assay includes the steps of

(i) providing a ubiquitin-conjugating system including the substrate polypeptide, ubiquitin and a cell- or tissue-specific F-box polypeptide and/or other SCF protein, under conditions which promote ubiquitination of the substrate polypeptide;

(ii) contacting the ubiquitin-conjugating system with a candidate agent;

(iii) measuring the level of ubiquitination of the substrate polypeptide in the presence of the candidate agent; and

(iv) comparing the measured level of ubiquitination in the presence of the candidate agent with the level of ubiquitination of the substrate polypeptide in the absence of the candidate agent.

In the subject assay, a statistically significant decrease in ubiquitination of the substrate polypeptide in the presence of the candidate agent is indicative of an inhibitor of cell- or tissue-specific F-box protein-mediated ubiquitination. The ubiquitin-conjugating system can be, e.g., a reconstituted protein mixture, a cell lysate or a whole cell.

The ubiquitin-conjugating system can also include an E2 ubiquitin conjugating enzyme, a cullins protein, a Skp1 protein, and/or an Rbx1 protein. The ubiquitin can be provided in such forms as (i) an unconjugated ubiquitin, in which case the ubiquitin-conjugating system further comprises an E1 ubiquitin-activating enzyme (E1), an E2 ubiquitin-conjugating enzyme (E2), and adenosine triphosphate; (ii) an activated E1:ubiquitin complex, in which case the ubiquitin-conjugating system further comprises an E2; (iii) an activated E2:ubiquitin complex; and/or (iv) an activated E3:ubiquitin complex, e.g., with one or more of the cell- or tissue-specific F-box proteins identified herein.

Preferred embodiments of the subject assay utilize atrophin-1 as the cell- or tissue-specific F-box protein, e.g., a vertebrate atrophin-1, more preferably a mammalian atrophin-1 (such as shown in FIG. 5B), and even more preferably a human atrophin-1 polypeptide.

In certain embodiments of the subject assay, at least one of the ubiquitin and the substrate polypeptides include a detectable label, and the level of ubiquitination of the substrate polypeptide is quantified by detecting the label in at least one of the substrate polypeptide, the ubiquitin, and ubiquitin-conjugated substrate polypeptide. For illustrative purposes, the label group can be a radioisotope, a fluorescent compound, an enzyme, or an enzyme co-factor. In one embodiment, the detectable label includes a polypeptide having a measurable activity, e.g., an enzymatic activity, and the substrate polypeptide is fusion protein including the detectable label. Preferred substrate polypeptides are regulatory components of muscle cells or components of the myofibrillar apparatus.

In other embodiments, the amount of ubiquitination of the substrate polypeptide is quantified by an immunoassay, e.g., using antibodies for ubiquitin, the substrate polypeptide and/or a heterologous label. In other embodiments, the amount of ubiquitination of the substrate polypeptide can be quantified by chromatography or electrophoresis.

In still other embodiments, the ubiquitin-conjugating system is a host cell expressing the substrate polypeptide and a cell- or tissue-specific F-box protein, e.g., atrophin-1, preferably one of the two being recombinantly produced by the cell. The ubiquitination of the substrate polypepeide polypeptide by the SCF complex can be detected, in addition to such direct means as described above, by the expression of a reporter gene under transcriptional control of a substrate responsive element. Accordingly, in another embodiment of the subject assay, inhibitors of ubiquitin-mediated proteolysis of an substrate polypeptide are identified by such steps as:

(i) providing a eukaryotic cell expressing a substrate polypeptide which inhibits transcriptional activation of a Rel transcription factor, an SCF complex containing a cell- or tissue-specific F-box protein which ubiquitinates the substrate polypeptide, and harboring a reporter gene under transcriptional control of a substrate polypeptide responsive element;

(ii) contacting the cell with a candidate agent;

(iii) measuring the level of expression of the reporter gene in the presence of the candidate agent; and

(iv) comparing the measured level of reporter gene expression in the presence of the candidate agent with reporter gene expression in the absence of the candidate agent,

wherein a statistically significant decrease in reporter gene expression in the presence of the candidate agent is indicative of an inhibitor of ubiquitination of the substrate polypeptide.

In yet another embodiment, the subject assay can be derived to identify inhibitors of an interaction between a substrate polypeptide and a cell- or tissue-specific F-box protein, rather than ubiquitination per se. Such assays can include the steps of:

(i) providing a reaction system including the substrate polypeptide and an SCF complex containing a cell-ortissue-specific F-box protein, under conditions wherein the substrate polypeptide and the F-box protein interact;

(ii) contacting the reaction system with a candidate agent;

(iii) measuring formation of complexes containing the substrate polypeptide and the F-box protein in the presence of the candidate agent; and

(iv) comparing the measured formation of complexes in the presence of the candidate agent with complexes formed in the absence of the candidate agent,

wherein a statistically significant decrease in the formation of complexes in the presence of the candidate agent is indicative of an inhibitor of the interaction of the substrate polypeptide and the F-box protein. The reaction system can be a reconstituted protein mixture, a cell lysate or a whole cell. In the instance of the latter, one preferred embodiment provides an interaction trap system including the substrate polypeptide and the F-box protein as bait and prey fusion proteins.

In each of the above embodiments, the substrate polypeptide can be, for example, selected from a group consisting of regulatory componenets of muscle cells or components of the myofibrillar apparatus. In some embodiments, the substrate polypeptide can be phosphorylated at sites which promote ubiquitination by the SCF protein complex.

In any embodiment of the subject assays, one or more of the compounds identified as inhibitors of the cell- or tissue-specific F-box protein-mediated ubiquitination can be formulated as a pharmaceutical preparation, e.g., for further in vivo testing and therapeutic use.

Yet another aspect of the present invention relates to diagnostic assays for determining, in the context of cells isolated from a patient, the level of a cell- or tissue-specific F-box MRNA transcript, cell- or tissue-specific F-box protein and/or cell- or tissue-specific F-box protein activity, which level can be a useful diagnostic/prognostic marker for risk assessment and phenotyping cell and tissue samples. As described herein, the subject assay provides a method for determining if an animal is at risk for a muscle wasting disorder characterized by aberrant cell proliferation, differentiation, apoptosis, and/or protein degredation and also may be used for prognostic purposes when such aberrant cell phenotypes are known.

The subject method can be used for diagnosing a muscle wasting disorder in a patient, comprising: (i) ascertaining the level of expression of an F-box polypeptide comprising the sequence set forth in FIG. 5B in a sample of muscle cells from the patient; and (ii) diagnosing the presence or absence of a muscle wasting disorder utilizing, at least in part, the ascertained level of expression or activity of the F-box polypeptide; wherein an increased level of expression of the F-box polypeptide or F-box polypeptide-dependent ubiquitination activity in the sample, relative to a control sample of non-muscle cells, correlates with the presence of a muscle wasting disorder.

Another aspect of the invention features a method for treating a patient suffering from a muscle wasting disorder comprising administering to the pateint an amount of an atrophin-1 inhibitor effective to inhibit the expression and/or activity of atrophin-1. The method is preferably used to treat patients wherein the muscle wasting disorder is associated with chachexia and other muscle wasting, e.g., cachexia secondary to infection or malignancy, cachexia secondary to human acquired immune deficiency syndrome (ADS), AIDS, ARC (ADS related complex); rheumatoid arthritis, rheumatoid spondylitis, osteoarthritis, gouty arthritis and other arthritic conditions; sepsis, septic shock, endotoxic shock, gram negative sepsis, toxic shock syndrome, adult respiratory distress syndrome, cerebral malaria, chronic pulmonary inflammatory disease, silicosis, pulmonary sarcoidosis, bone resorption diseases, reperfusion injury, graft vs. host reaction, allograft rejections, Crohn's disease, ulcerative colitis, or pyresis, in addition to a number of autoimmune diseases, such as multiple sclerosis, autoimmune diabetes and systemic lupus erythematosis. In addition to treatment of diseases associated with muscle wasting, inhibitors of atrophin-1 could be useful in maintaining muscle mass in bedridden patients or in space personnel in whom muscle wasting due to the prolonged microgravity environment is a major problem. Inhibitors of atrohpin-1 may also be useful for promoting muscle formation, stimulating proliferating of muscle stem cells, increasing muscle mass, e.g., production of livestock animals with increased muscle mass, etc.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press:1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### BRIEF DESCRIPTION OF THE FIGURES

FIG. 1 shows a diagrammatic overview of protein degradation via the ubiquitin pathway.

FIG. 2 shows a diagram of the SCF ubiquitin-protein ligase complex (E3).

FIG. 3 shows Northern blot analysis of total RNA isolated from normal or atrophying rodent muscle which was probed with a truncated human atrophin-1 cDNA. mRNA for atrophin-1 is increased in atrophying muscles due to many causes.

FIG. 4 shows Northern blot analysis of total RNA isolated from normal or atrophying mouse muscle due to food deprivation, and probed with a full-length murine atrophin-1 cDNA. MRNA for atrophin-1 is specifically expressed in muscle.

FIG. 5 shows nucleotide and amino acid sequences of atrophin-1. A, shows the nucleotide sequence of the mouse atrohpin-1 gene. B, shows the deduced amino acid sequence of mouse atrophin-1 protein. The F-box motif is underlined. C, shows a schematic representation of the atrophin-1 protein. The box represents the F-box motif.

### DETAILED DESCRIPTION OF THE INVENTION

1. General

Whether a muscle grows or atrophies depends on the overall balance between its rate of protein synthesis and breakdown. It is now clear that increased protein breakdown is the primary cause of the rapid loss of muscle mass and myofibrillar proteins that occurs upon denervation or disuse and in many systemic diseases, including diabetes, sepsis, hyperthyroidism, cancer cachexia, or fasting. Greater knowledge about the mechanisms and regulation of proteolysis in muscle is essential if we are to develop rational therapies to combat muscle wasting.

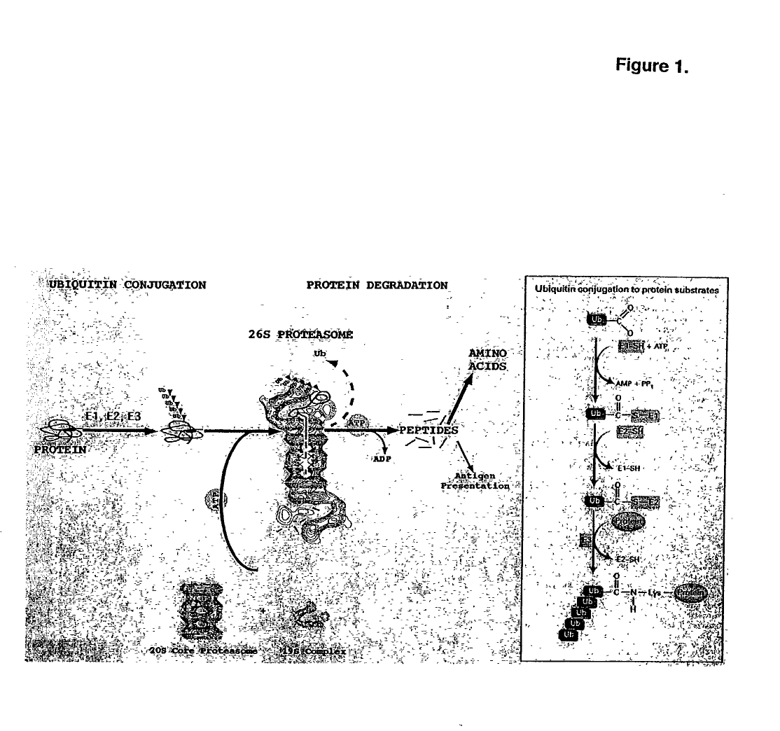

Enhancement of proteolysis in atrophying muscles results mainly from activation of the degradative pathway involving ubiquitin (Ub) and the proteasome particle. In this ATP-dependent pathway, proteins are marked for degradation by linking them covalently to a chain of Ub molecules, which targets the proteins for rapid breakdown by the 26S proteasome (FIG. 1). Ub conjugation is a multi-step process involving first activation of Ub by the enzyme E1 and then linkage of the activated Ub to one of the cells Ub-carrier proteins (termed E2s). Finally, the Ub is transferred to the substrate by one of the cells many Ub-protein ligases (termed E3s) (FIG. 1). Generally, an E2-E3 pair functions together in the ubiquitination of specific proteins.

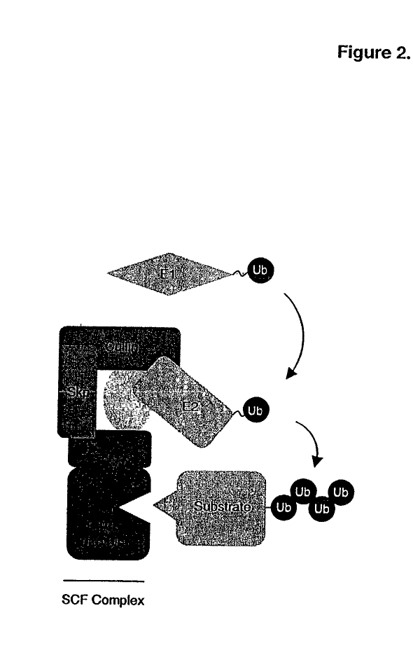

Recently, a new class of Ub-protein ligases has been described, called SCF complexes (FIG. 2). These multisubunit enzymes contain one protein that binds an E2 (i.e. the cullin), one protein that serves as a scaffold (the skp protein) and a subunit which recognizes the substrate to be ubiquitinated (the F-box component). The F-box motif is the conserved protein sequence within this substrate receptor that binds to the skp protein, forming the SCF complex. A large family of F-box proteins have been identified based on this conserved motif, but the functions and substrates of these ubiquitination enzymes has lagged far behind their genetic and biochemical elucidation. This invention concerns a new F-box protein that is specific to muscle and is expressed in increased amounts during various types of with muscle atrophy.

In several different animal models of atrophy, it has been found that muscles exhibit a common series of adaptations that indicate an activation of the Ub-proteasome pathway. For example, these muscles have an increased content of Ub-protein conjugates (the critical intermediates in this pathway) and of mRNA encoding Ub, certain ubiquitination enzymes (e.g., E2₁₄ₖ and E3α) and multiple proteasome subunits. These studies have identified several genes (e.g. genes for Ub, E²14k), whose expression rises in all these models of muscle wasting, despite the general fall in mRNA content when muscles atrophy. Presumably there are many other genes whose increased expression is crucial for the loss of muscle mass and functional capacity.

The present invention relates to a new class of vertebrate F-box proteins, referred to herein as atrophins, that are specific to muscle and, in the case of atrophin-1, is expressed in increased amounts during various types of muscle atrophy. In particular, the present invention is based at least in part on the isolation of a full length cDNA encoding a protein containing an F-box, e.g., a protein including a motif found in many ubiquitin-protein ligases (E3s) involved in intracellular proteolysis. Based on both biochemical/biological data, and identity of an F-box motif, the subject protein is understood to be involved in the ubiquitinylation of proteins in vertebrate cells.

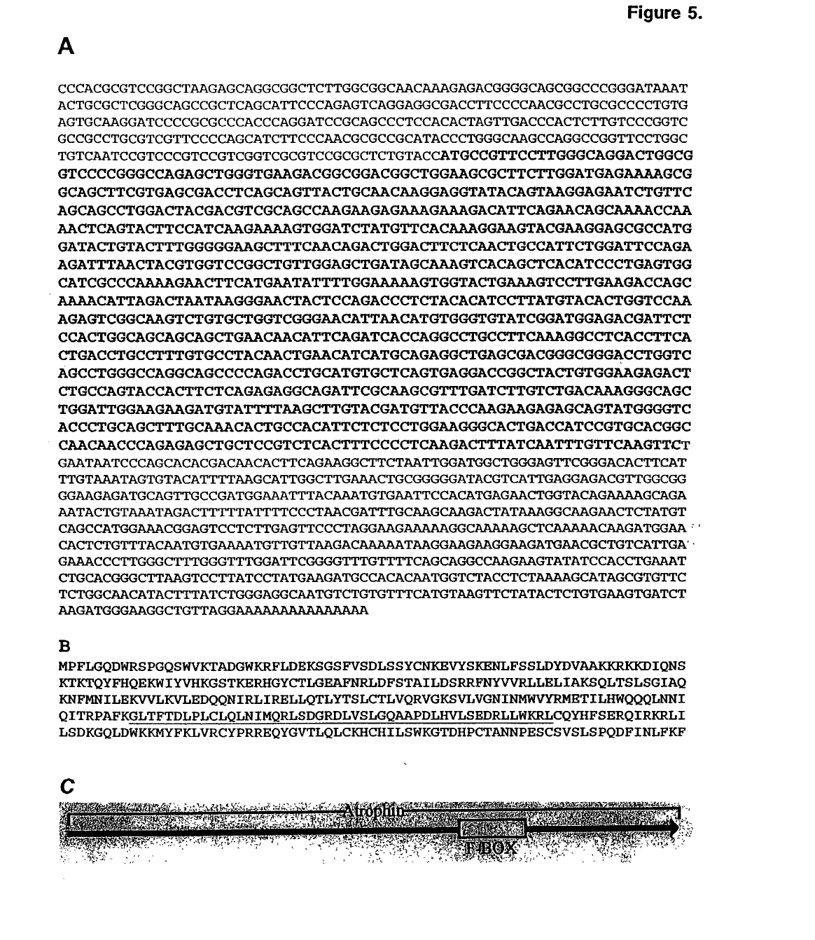

The DNA sequence obtained for the atrophin-1 clone is given in FIG. 5A. The sequence for the ˜40 kDa polypeptide encoded by the atrophin-1 gene is given in FIG. 5B. The F box is a 48 amino acid region corresponding to residues 222-269 of FIG. 5B.

Accordingly, the present invention provides nucleic acids and the proteins which function in the ubiquitinylation of substrate proteins. The invention also provides methods for modulating protein degradation, assays for identifying compounds which modulate protein degradation, methods for treating disorders associated with aberrant protein degradation, diagnostic and prognostic assays for determining whether a subject is at risk of developing a disorder associated with an aberrant protein degradation. For example, inhibitors of the atrophin-1 enzyme could be useful in combating a number of diseases including chachexia and other muscle wasting, e.g., cachexia secondary to infection or malignancy, cachexia secondary to human acquired immune deficiency syndrome (ADS), AIDS, ARC (ADS related complex); rheumatoid arthritis, rheumatoid spondylitis, osteoarthritis, gouty arthritis and other arthritic conditions; sepsis, septic shock, endotoxic shock, gram negative sepsis, toxic shock syndrome, adult respiratory distress syndrome, cerebral malaria, chronic pulmonary inflammatory disease, silicosis, pulmonary sarcoidosis, bone resorption diseases, reperfusion injury, graft vs. host reaction, allograft rejections, Crohn's disease, ulcerative colitis, or pyresis, in addition to a number of autoimmune diseases, such as multiple sclerosis, autoimmune diabetes and systemic lupus erythematosis. In addition to treatment of diseases associated with muscle wasting, inhibitors of atrophin-1 could be useful in maintaining muscle mass in bedridden patients or in space personnel in whom muscle wasting due to the prolonged microgravity environment is a major problem. Inhibitors of atrohpin-1 may also be useful for promoting muscle formation, stimulating proliferating of muscle stem cells, increasing muscle mass, e.g., production of livestock animals with increased muscle mass, etc.

2. Definitions

For convenience, certain terms employed in the specification, examples, and appended claims are collected here.

The term “aberrant activity”, as applied to an activity of an F-box polypeptide, e.g., atrophin-1, refers to an activity which differs from the activity of the wild-type or native form of the protein or because its level of expression is elevated or depressed as compared to the level occuring in a normal cell under normal physiological conditions. An activity of a protein can be aberrant because it is unregulated, e.g., constitutively activated or inactivated, relative to its normal state. An aberrant activity can also be a change in an activity. For example an aberrant protein can interact with a different protein relative to its native counterpart. A cell can also have an aberrant F-box polypeptide activity due to overexpression or underexpression of the gene encoding an F-box polypeptide.

The term “agonist” as used herein, refers to a molecule which augments formation of a protein complex with an F-box protein, or which, when bound to an F-box containing complex or a molecule in the complex, increases the amount of, or prolongs the duration of, the activity of the F-box protein. Agonists may include proteins, nucleic acids, carbohydrates, or any other molecules that bind to an F-box containing complex or molecule of the complex. Agonists also include a functional peptide or peptide fragment derived from an F-box protein or a protein which binds to an F-box protein, or it may include the F-box protein or a protein which binds to an F-box protein themselves. Peptide mimetics, synthetic molecules with physical structures designed to mimic structural features of particular peptides, may serve as agonists. The stimulation may be direct, or indirect, or by a competitive or non-competitive mechanism.

As used herein the term “animal” refers to mammals, preferably mammals such as humans.

The term “antagonist”, as used herein, refers to a molecule which, when bound to an F-box protein, an F-box protein containing complex, or a molecule in the complex, decreases the amount of or duration of the activity of the F-box protein, the F-box protein containing complex, or a protein member thereof, or decreases F-box complex formation. Antagonists include compounds which directly inhibit the activity of an F-box protein, e.g., atrohpin-1, by inhibiting the ligase activity of an atrophin-1 containing SCF complex through chemical alteration of an active site cysteine residue of a member of the SCF complex. Antagonists may include proteins including antibodies that compete for binding at a binding region of an F-box complex member, nucleic acids including anti-sense molecules that arrest expression of an F-box complex member at the genetic level, carbohydrates, or any other molecules that bind to a mammalian, preferably human, form of an F-box protein, to an extent efficient for preventing F-box complex formation or activity. Anatagonists also include dominant negative mutatnts, e.g. a member of an atrophin-1 containing SCF complex which contains a mutated active site. Antagonists further include a peptide or peptide fragment derived from an F-box protein or member of an F-box containing protein complex, but will not include the full length sequence of the wild-type molecule. Peptide mimetics, synthetic molecules with physical structures designed to mimic structural features of particular peptides, may serve as antagonists. The inhibition may be direct, or indirect, or by a competitive or non-competitive mechanism.

“Atrophin-1” refers to a murine F-box protein which is specifically expressed in muscle cells that has a sequence that is either the sequence of murine atrophin-1 or a sequence that shares substantial sequence identity therewith, including mammalian (e.g. human) homologs thereof. The sequence of murine atrophin-1 is provided below:

MPFLGQDWRSPGQSWVKTADGWKRFLDEKSGSFVSDLSSYCNKEVYSKEN
LFSSLDYDVAAKKRKKDIQNSKTKTQYFHQEKWIYVHKGSTKERHGYCTL
GEAFNRLDFSTAILDSRRFNYVVRLLELIAKSQLTSLSGIAQKNFMNILE
KVVLKVLEDQQNIRLIRELLQTLYTSLCTLVQRVGKSVLVGNINMWVYRM
ETILHWQQQLNNIQITRPAFKGLTFTDLPLCLQLNIMQRLSDGRDLVSLG
QAAPDLHVLSEDRLLWKRLCQYHFSERQIRKRLILSDKGQLDWKKM
YFKLVRCYPRREQYGVTLQLCKHCHILSWKGTDHPCTANNPESCSVSLSP
QDFINLFKF

The F-box sequence is underlined.

The terms “bait” or “bait protein” refer to a polypeptide which is used as a target to find other proteins which may associate with it. Typically, a bait protein is tagged or immobilized so as to allow easy isolation of complexes involving the bait protein.

The term “binding” refers to a stable association between two molecules, in the present case between an F-box protein and a binding partner, such as another E3 polypeptide, an E2 conjugating enzyme, Skp1, cdc53, Rbx1, or a protein substrate, due to, for example, electrostatic, hydrophobic, ionic and/or hydrogen-bond interactions under physiological conditions).

As used herein the term “bioactive fragment of an F-box protein” refers to a fragment of a full-length F-box protein, wherein the fragment specifically mimics or antagonizes the activity of a wild-type F-box protein. The bioactive fragment preferably is a fragment capable of binding to a second protein, e.g., another protein involved in ubiquitin conjugation.

“Biological activity” or “bioactivity” or “activity” or “biological function”, which are used interchangeably, for the purposes herein means an effector or antigenic function that is directly or indirectly performed by an F-box polypeptide (whether in its native or denatured conformation), or by any subsequence thereof. Biological activities include binding to another protein, such as another E3 polypeptide, an E1, an E2 conjugating enzyme, a skp1 protein, a cdc53 protein, a Rbx1 protein, and/or a substrate protein. In particular, the biological activity of an F-box polypeptide of the invention can be binding of the protein to a cullins protein, a skp1 protein, a ubiquitin conjugating enzyme or a substrate protein. The biological activity of an F-box polypeptide can also include the ability to mediate ubiquitination of a substrate protein, such as when the F-box polypeptide is associated with other proteins, e.g., other components of an E3 complex and substrate proteins. The biological activity of an F-box polypeptide can also include: an ability to specifically modulate protein degradation in a muscle cell. Biologically active F-box polypeptides include polypeptides having both an effector and antigenic function, or only one of such functions. The term “F-box protein” also includes antagonist polypeptides and native F-box proteins, provided that such antagonists include an epitope of a native F-box protein.

The term “cdc53” is used interchangeably herein with the term “cullins” when referring to a vertebrate homolog of the yeast cdc53 protein. The term “cullins polypeptide” or “cullins protein”, refers to a member of the cullins family, e.g., an one of cul-1, -2, -3, -4, -5, or -6.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

“Cell- or tissue-specific F-box protein” refers to a protein containing an F-box motif which is expressed at a substantially higher level in one cell or tissue type as compared to another cell or tissue type. By “substantially higher level” it is meant that the F-box protein is expressed in one tissue at least 2-fold and preferably at least 5-fold or 10-fold higher than the expression level of the same protein in another cell or tissue type. The level of the protein expression may be determined by measuring the level of mRNA that encodes the protein or by measuring the level of the protein itself. Preferably, but not essentially, a cell- or tissue-specific F-box protein is detectably expressed in only one type of cell or tissue and is not detectably expressed in any other cell or tissue type (by art recognized methods, such as Northern blot, Western blot, etc.). The definition of cell- or tissue-specific F-box protein is meant to include F-box containing proteins which are specifically expressed in one species and not in another (e.g. expressed in yeast but not in humans, or expressed at a substantially higher level in yeast than in humans, etc.), proteins which are expressed in one tissue but not in another (e.g. expressed in muscle but not in kidney tissues, or expressed at a substantially higher level in muscle tissue than in kidney tissue, etc.) and proteins which are expressed in one cell type but not in another (e.g. expressed in smooth muscle cells but not in endothelial cells, or expressed at a substantially higher level in smooth muscle cells than in endothelial cells, etc.).

The term “charged lysate” refers to cell lysates which have been spiked with exogenous, e.g., purified, semi-purified and/or recombinant, forms of one or more components of an F-box-dependent ubiquitin-conjugating system, or a substrate protein thereof. The lysate can be charged after the whole cells have been harvested and lysed, or alternatively, by virtue of the cell from which the lysate is generated expressing a recombinant form of one or more of the conjugating system components.

A “chimeric protein” or “fusion protein” is a fusion of a first amino acid sequence encoding a polypeptide with a second amino acid sequence defining a domain foreign to and not substantially homologous with any domain of the protein. A chimeric protein may present a foreign domain which is found (albeit in a different protein) in an organism which also expresses the first protein, or it may be an “interspecies”, “intergenic”, etc. fusion of protein structures expressed by different kinds of organisms.

The term “component of a ubiquitin-conjugation pathway”, as used herein, refers to a component which can participate in the ubiquitination of a substrate protein either in vivo or in vitro. Exemplary components of a ubiquitin-conjugation pathway include ubiquitin, an E1, an E2, an F-box protein or protein complex, a substrate protein, and the like.

The terms “compound”, “test compound” and “molecule” are used herein interchangeably and are meant to include, but are not limited to, peptides, nucleic acids, carbohydrates, small organic molecules, and natural product extract libraries.

The phrases “conserved residue” “or conservative amino acid substitution” refer to groupings of amino acids on the basis of certain common properties. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer., Principles of Protein Structure, Springer-Verlag). Examples of amino acid groups defined in this manner include:

(i) a charged group, consisting of Glu and Asp, Lys, Arg and His,

(ii) a positively-charged group, consisting of Lys, Arg and His,

(iii) a negatively-charged group, consisting of Glu and Asp,

(iv) an aromatic group, consisting of Phe, Tyr and Trp,

(v) a nitrogen ring group, consisting of His and Trp,

(vi) a large aliphatic nonpolar group, consisting of Val, Leu and Ile,

(vii) a slightly-polar group, consisting of Met and Cys,

(viii) a small-residue group, consisting of Ser, Thr, Asp, Asn, Gly, Ala, Glu, Gln and Pro,

(ix) an aliphatic group consisting of Val, Leu, Ile, Met and Cys, and

(x) a small hydroxyl group consisting of Ser and Thr.

In addition to the groups presented above, each amino acid residue may form its own group, and the group formed by an individual amino acid may be referred to simply by the one and/or three letter abbreviation for that amino acid commonly used in the art.

The term “cullins polypeptide” or “cullins protein”, refers to a member of the cullins family, e.g., any one of cul-1, -2, -3, -4, -5, or -6.

The terms “destruction box sequence” or “destruction box motif” refer to the amino acid consensus sequence RxxLxxxxN which is essential for the ubiquitin mediated degradation of some cell cycle related proteins (Glotzer et al. (1991) Nature 349:132-138). It is thought that the destruction box sequence acts as a recognition element between the protein and its specific ubiquitination machinery.

The term “DNA sequence encoding a polypeptide” may refer to one or more genes within a particular individual. As is well known in the art, genes for a particular polypeptide may exist in single or multiple copies within the genome of an individual. Such duplicate genes may be identical or may have certain modifications, including nucleotide substitutions, additions or deletions, which all still code for polypeptides having substantially the same activity. Moreover, certain differences in nucleotide sequences may exist between individual organisms, which are called alleles. Such allelic differences may or may not result in differences in amino acid sequence of the encoded polypeptide yet still encode a protein with the same biological activity.

The term “domain” as used herein refers to a region within a protein that comprises a particular structure or function different from that of other sections of the molecule.

The term “E3 complex” refers to a protein complex including the subject F-box proteins, which protein complex augments or otherwise facilitates the ubiquitination of a protein. In preferred embodiments, the E3 complex has cell- or tissue-specific activity.

As used herein “F-box” or “F-box motif” or “F-box domain” refer to an amino acid consensus sequence as defined by:

ZJXZPZUZZXXZZXXXXXXXZZXZXXVXBBZXXZZXXXXZOXXZ

wherein Z is a nonpolar amino acid residue (ala, val, leu, iso, pro, phe, met, trp), X is any amino acid residue, B is a basic amino acid residue (lys, arg, his), U is an acidic amino acid residue (asp, glu), O is an aromatic amino acid residue (phe, tyr, trp), J is either serine or threonine (ser, thr), and P and V are the standard single letter representations for proline and valine, respectively (Craig and Tyers, Prog. Biophys. & Mol. Biol. 72: 299-328 (1999)). An “F-box protein” refers to a polypeptide which contains an F-box motif. Various types of searches may be used to identifying proteins which may contain an F-box motif. For example, on-line databases such as GenBank or SwissProt can be searched, either with an entire sequence of an F-box-containing protein, or with a consensus F-box motif sequence. Various search algorithms and/or programs may be used, including FASTA, BLAST or ENTREZ. FASTA and BLAST are available as a part of the GCG sequence analysis package (University of Wisconsin, Madison, Wis.). ENTREZ is available through the National Center for Biotechnology Information, National Library of Medicine, National Institutes of Health, Bethesda, Md.

As used herein “F-box-dependent ubiquitination” refers to the conjugation of ubiquitin to a protein by a mechanism which requires an F-box protein or an F-box-containing protein complex, e.g., which is dependent on the presence of an F-box protein.

The term “F-box therapeutic” refers to various forms of F-box polypeptides, as well as peptidomimetics, small molecules, nucleic acids, and antibodies, which can modulate at least one activity of an F-box protein, e.g., binding to another protein, by mimicking or potentiating (agonizing) or inhibiting (antagonizing) the effects of a naturally-occurring F-box protein, inhibiting an enzymatic activity of a ubiquitin ligase activity, or inhibits expression of an F-box protein. An F-box therapeutic which mimics or potentiates the activity of a wild-type F-box protein is an “F-box agonist”. Conversely, an F-box therapeutic which inhibits the activity of a wild-type F-box protein is an “F-box antagonist”.

As used herein, the term “gene” or “recombinant gene” refers to a nucleic acid comprising an open reading frame encoding a polypeptide of the present invention, including both exon and (optionally) intron sequences. A “recombinant gene” refers to nucleic acid encoding a polypeptide and comprising exon coding sequences, though it may optionally include intron sequences derived from a chromosomal gene. The term “intron” refers to a DNA sequence present in a given gene which is not translated into protein and is generally found between exons.

“Homology” or “identity” or “similarity” refers to sequence similarity between two peptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 40% identity, though preferably less than 25% identity with a sequence of the present invention.

The term “homology” describes a mathematically based comparison of sequence similarities which is used to identify genes or proteins with similar functions or motifs. The nucleic acid and protein sequences of the present invention may be used as a “query sequence” to perform a search against public databases to, for example, identify other family members, related sequences or homologs. Such searches can be performed using the NBLAST and XBLAST programs (version 2.0) of Altschul, et al. (1990) J Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used. See http://www.ncbi.nlm.nih.gov.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

The term “isolated”, as used herein with reference to an F-box protein or F-box protein containing complex, refers to an F-box protein or F-box protein containing complex that is essentially free from contaminating proteins that normally would be present in cellular milieu in which the protein occurs or the complex forms endogenously. Thus, an isolated an F-box protein or F-box protein containing complex is isolated from cellular components that normally would “contaminate” or interfere with the study of the protein or protein complex in isolation, for instance while screening for modulators thereof. It is to be understood, however, that such an “isolated” protein or protein complex may incorporate other proteins the modulation of which, by the F-box protein or F-box protein containing complex, is being investigated. Such additional proteins may, for instance, include ubiquitin, an E1, an E2, a substrate protein, and the like.

The term “isolated” as also used herein with respect to nucleic acids, such as DNA or RNA, refers to molecules separated from other DNAs, or RNAs, respectively, that are present in the natural source of the macromolecule. For example, isolated nucleic acids encoding a polypeptide preferably include no more than 10 kilobases (kb) of nucleic acid sequence which naturally immediately flanks a particular gene in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state.

Polypeptides referred to herein as “mammalian homologs” of a protein refers to other mammalian paralogs, or other mammalian orthologs.

The term “motif” as used herein refers to an amino acid sequence that is commonly found in a protein of a particular structure or function. Typically a consensus sequence is defined to represent a particular motif. The consensus sequence need not be strictly defined and may contain positions of variability, degeneracy, variability of length, etc. The consensus sequence may be used to search a database to identify other proteins that may have a similar structure or function due to the presence of the motif in its amino acid sequence. For example, on-line databases such as GenBank or SwissProt can be searched with a consensus sequence in order to identify other proteins containing a particular motif. Various search algorithms and/or programs may be used, including FASTA, BLAST or ENTREZ. FASTA and BLAST are available as a part of the GCG sequence analysis package (University of Wisconsin, Madison, Wis.). ENTREZ is available through the National Center for Biotechnology Information, National Library of Medicine, National Institutes of Health, Bethesda, Md.

The “non-human animals” of the invention include vertebrates such as rodents, non-human primates, sheep, dog, cow, chickens, amphibians, reptiles, etc. Preferred non-human animals are selected from the rodent family including rat and mouse, most preferably mouse, though transgenic amphibians, such as members of the Xenopus genus, and transgenic chickens can also provide important tools for understanding, for example, embryogenesis and tissue patterning. The term “chimeric animal” is used herein to refer to animals in which the recombinant gene is found, or in which the recombinant is expressed in some but not all cells of the animal. The term “tissue-specific chimeric animal” indicates that the recombinant gene is present and/or expressed in some tissues but not others.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides.

The terms peptides, proteins and polypeptides are used interchangeably herein.

The terms “PEST sequence” or “PEST motif” refer to regions of proteins that are rich in proline, aspartate, glutamate, serine and threonine residues. PEST sequences seem to act as degradation signals for a variety of proteins via the ubiquitin pathway. It is thought that PEST regions act as recognition elements between a protein and its specific ubiquitination machinery.

As used herein, “phenotype” refers to the entire physical, biochemical, and physiological makeup of a cell, e.g., having any one trait or any group of traits.

The term “purified protein” refers to a preparation of a protein or proteins which are preferably isolated from, or otherwise substantially free of, other proteins normally associated with the protein(s) in a cell or cell lysate. The term “substantially free of other cellular proteins” (also referred to herein as “contaminating proteins”) is defined as encompassing individual preparations of each of the component proteins comprising less than 20% (by dry weight) contaminating protein, and preferably comprises less than 5% contaminating protein. Functional forms of each of the component proteins can be prepared as purified preparations by using a cloned gene. By “purified”, it is meant, when referring to component protein preparations used to generate a reconstituted protein mixture, that the indicated molecule is present in the substantial absence of other biological macromolecules, such as other proteins (particularly other proteins which may substantially mask, diminish, confuse or alter the characteristics of the component proteins either as purified preparations or in their function in the subject reconstituted mixture). The term “purified” as used herein preferably means at least 80% by dry weight, more preferably in the range of 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above. “Isolated” and “purified” do not encompass either protein in its native state (e.g. as a part of a cell), or as part of a cell lysate, or that have been separated into components (e.g., in an acrylamide gel) but not obtained either as pure (e.g. lacking contaminating proteins) substances or solutions. The term isolated as used herein also refers to a component protein that is substantially free of cellular material or culture medium when produced by recombinant DNA techniques, or chemical precursors or other chemicals when chemically synthesized.

The term “recombinant protein” refers to a protein of the present invention which is produced by recombinant DNA techniques, wherein generally DNA encoding the expressed protein is inserted into a suitable expression vector which is in turn used to transform a host cell to produce the heterologous protein. Moreover, the phrase “derived from”, with respect to a recombinant gene encoding the recombinant protein is meant to include within the meaning of “recombinant protein” those proteins having an amino acid sequence of a native protein, or an amino acid sequence similar thereto which is generated by mutations including substitutions and deletions of a naturally occurring protein.

As used herein, a “reporter gene construct” is a nucleic acid that includes a “reporter gene” operatively linked to a transcriptional regulatory sequence. Transcription of the reporter gene is controlled by these sequences. The activity of at least one or more of these control sequences is directly or indirectly regulated by a signal transduction pathway involving a ubiquitin substrate protein of the subject F-box proteins. The transcriptional regulatory sequences can include a promoter and other regulatory regions, such as enhancer sequences, that modulate the level of expression of a reporter gene in response to the level of a substrate protein.

The term “SCF complex” refers to a multi-subunit ubiquitin ligase comprising a Skp1 subunit, a cullins subunit, an Rbx1 subunit and an F-box protein subunit or homologs thereof. The terms “SCF complex”, “E3” and “ligase” or “ubiquitin ligase” are used interchangeably herein.

By “semi-purified”, with respect to protein preparations, it is meant that the proteins have been previously separated from other cellular or viral proteins. For instance, in contrast to whole cell lysates, the proteins of reconstituted conjugation system, together with the substrate protein, can be present in the mixture to at least 50% purity relative to all other proteins in the mixture, more preferably are present at least 75% purity, and even more preferably are present at 90-95% purity.

The term “semi-purified cell extract” or, alternatively, “fractionated lysate”, as used herein, refers to a cell lysate which has been treated so as to substantially remove at least one component of the whole cell lysate, or to substantially enrich at least one component of the whole cell lysate. “Substantially remove”, as used herein, means to remove at least 10%, more preferably at least 50%, and still more preferably at least 80%, of the component of the whole cell lysate. “Substantially enrich”, as used herein, means to enrich by at least 10%, more preferably by at least 30%, and still more preferably at least about 50%, at least one component of the whole cell lysate compared to another component of the whole cell lysate. The component which is removed or enriched can be a component of a ubiquitin-conjugation pathway, e.g., ubiquitin, a substrate protein, an E1, an E2, or (an) F-box protein(s), and the like, or it can be a component which can interfere with a ubiquitin-binding assay, e.g., a protease. The term “semi-purified cell extract” is also intended to include the lysate from a cell, when the cell has been treated so as to have substantially more, or substantially less, of a given component than a control cell. For example, a cell which has been modified (by, e.g., recombinant DNA techniques) to produce none (or very little) of a component of a ubiquitin-conjugation pathway, will, upon cell lysis, yield a semi-purified cell extract.

“Small molecule” as used herein, is meant to refer to a composition, which has a molecular weight of less than about 5 kD and most preferably less than about 2.5 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention.

As used herein, the term “specifically hybridizes” refers to the ability of a nucleic acid probe/primer of the invention to hybridize to at least 15, 25, 50 or 100 consecutive nucleotides of a target gene sequence, or a sequence complementary thereto, or naturally occurring mutants thereof, such that it has less than 15%, preferably less than 10%, and more preferably less than 5% background hybridization to a cellular nucleic acid (e.g., mRNA or genomic DNA) other than the target gene.

As applied to polypeptides, “substantial sequence identity” means that two mammalian peptide sequences, when optimally aligned, such as by the programs GAP or BESTFIT using default gap which share at least 90 percent sequence identity, preferably at least 95 percent sequence identity, more preferably at least 99 percent sequence identity or more. Preferably, residue positions which are not identical differ by conservative amino acid substitutions. For example, the substitution of amino acids having similar chemical properties such as charge or polarity are not likely to effect the properties of a protein. Examples include glutamine for asparagine or glutamic acid for aspartic acid.

The term “substrate”, “substrate protein” or “target protein” refers to a protein, preferably a cellular protein, which can be ubiquitinated by an F-box protein-dependent reaction pathway. Preferred substrates of atrohpin-1 are regulatory components of muscle cells or components of the myofibrillar apparatus.

As used herein, the term “tissue-specific promoter” means a DNA sequence that serves as a promoter, i.e., regulates expression of a selected DNA sequence operably linked to the promoter, and which effects expression of the selected DNA sequence in specific cells of a tissue, such as cells of a urogenital origin, e.g. renal cells, or cells of a neural origin, e.g. neuronal cells. The term also covers so-called “leaky” promoters, which regulate expression of a selected DNA primarily in one tissue, but cause expression in other tissues as well.

As used herein, the term “transfection” means the introduction of a nucleic acid, e.g., an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of a polypeptide of the present invention or where anti-sense expression occurs from the transferred gene so that the expression of a naturally-occurring form of the gene is disrupted.

As used herein, the term “transgene” means a nucleic acid sequence, which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can include one or more transcriptional regulatory sequences and any other nucleic acid, such as introns, that may be necessary for optimal expression of a selected nucleic acid.

As used herein, a “transgenic animal” is any animal, preferably a non-human mammal, a bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of a protein, e.g. either agonistic or antagonistic forms. However, transgenic animals in which the recombinant gene is silent are also contemplated, as for example, the FLP or CRE recombinase dependent constructs described below.

“Transcriptional regulatory sequence” is a generic term used throughout the specification to refer to DNA sequences, such as initiation signals, enhancers, and promoters, which induce or control transcription of protein coding sequences with which they are operably linked. In preferred embodiments, transcription of a recombinant protein gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of the protein.

“Ub” refers to ubiquitin.

“Ubiquitination” refers to the activity of polypeptides capable of forming a thiol ester adduct, such as with the C-terminal carboxyl group of ubiquitin and transferring the ubiquitin to an ε-amino group in an acceptor protein by formation of an isopeptide bond, or some other covalent modification which links ubiquitin to a polypeptide chain, e.g., such as with regard to the activity of a “ubiquitin-conjugating enzyme” or “ubiquitin ligase”.

A “ubiquitination sequence” refers to a portion of a protein which is sufficient to cause F-box protein-mediated ubiquitination of the protein.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

A “WD-40 motif”, also referred to in the art as “β-transducin repeats” or “WD-40 repeats”, is roughly defined as a contiguous sequence of about 25 to 50 amino acids with relatively-well conserved sets of amino acids at the two ends (amino- and carboxyl-terminal) of the sequence (reviewed in Simon et al., Science 252:802-808 (1991) and Neer et al., Nature 371:297 (1994)). Conserved sets of at least one WD-40 repeat of a WD-40 repeat-containing protein typically contain conserved amino acids at certain positions. The amino-terminal set, comprised of two contiguous amino acids, often contains a Gly followed by a His. The carboxyl-terminal set, comprised of six to eight contiguous amino acids, typically contains an Asp at its first position, and a Trp followed by an Asp at its last two positions. A general formula for characterizing a WD40 repeat is

{X₆₋₉₄-[GH-X₂₃₋₄₁-WD]}_{N}

wherein X₆₋₉₄ represents from 6 to 94 contiguous amino acid residues, X₂₃₋₄₁ represents from 23 to 41 contiguous amino acid residues, and N represents an integer from 4-8 (Neer et al., Nature 371:297 (1994)). Other WD40 repeats will, however, be appreciated by those skilled in the art. The number of WD-40 repeats in a particular protein can range from two to more than eight.

The term “whole lysate” refers to a cell lysate which has not been manipulated, e.g. either fractionated, depleted or charged, beyond the step of merely lysing the cell to form the lysate. The term whole cell lysate does not, however, include lysates derived from cells which produce recombinant forms of one or more of the proteins required to constitute a ubiquitin-conjugating system for F-box-dependent ubiquitination of a substrate protein.

3. Cell- or Tissue-specific F-Box Nucleic Acids and Expression Vectors

As described below, one aspect of the invention pertains to isolated nucleic acid having a nucleotide sequence encoding a cell- or tissue-specific F-box protein, e.g., a vertebrate cell- or tissue-specific F-box protein, such as atrophin-1, and/or equivalents of such nucleic acids. The term nucleic acid as used herein is intended to include fragments and equivalents. The term equivalent is understood to include nucleotide sequences encoding functionally equivalent cell- or tissue-specific F-box proteins or functionally equivalent polypeptides which, for example, retain the ability to bind to another protein, such as another component of an SCF complex, such as skp1, cdc53. Rbx1, or a substrate protein. Equivalent nucleotide sequences will include sequences that differ by one or more nucleotide substitutions, additions or deletions, such as allelic variants; and will, therefore, include coding sequences that differ from the nucleotide sequence of the coding sequence shown in FIG. 5A e.g., due to the degeneracy of the genetic code. Equivalents will also include nucleotide sequences that hybridize under stringent conditions (i.e., equivalent to about 20-27° C. below the melting temperature (Tₘ) of the DNA duplex formed in about 1M salt) to the nucleotide sequence of an coding sequence represented in FIG. 5A. In one embodiment, equivalents will further include nucleic acid sequences derived from and evolutionarily related to a nucleotide sequence shown in FIG. 5A.

Moreover, it will be generally appreciated that, under certain circumstances, it may be advantageous to provide homologs of the subject cell- or tissue-specific F-box proteins, which homologs function in a limited capacity as one of either an agonist (mimetic) or an antagonist in order to promote or inhibit only a subset of the biological activities of the naturally-occurring form of the protein. Thus, specific biological effects can be elicited by treatment with a homolog of limited function, and with fewer side effects relative to treatment with agonists or antagonists which are directed to all of a cell- or tissue-specific F-box protein's biological activities. For instance, antagonistic homologs can be generated which interfere with the ability of the wild-type (“authentic”) cell- or tissue-specific F-box protein to associate with other proteins in the ubiquitination pathway, but which do not substantially interfere with the formation of complexes between the native cell- or tissue-specific F-box protein and other cellular proteins, such as may be involved in other regulatory mechanisms of the cell.

Polypeptides referred to herein as cell- or tissue-specific F-box polypeptides preferably have an amino acid sequence corresponding to all or a portion of the cell- or tissue-specific F-box amino acid sequence shown in FIG. 5B, or are homologous with this protein, such as other human paralogs, or mammalian orthologs.

In general, the biological activity of a cell- or tissue-specific F-box polypeptide will be characterized as including the ability, in the presence of other required proteins, to mediate and/or catalyze the transfer a ubiquitin molecule from a relevant ubiquitin conjugating enzyme (UBC) to a lysine residue of its substrate protein. The above notwithstanding, the biological activity of a cell- or tissue-specific F-box polypeptide may be characterized by one or more of the following attributes: an ability to regulate the cell-cycle of an eukaryotic cell, especially a mammalian cell (e.g., of a human cell); an ability to modulate proliferation/cell growth of a eukaryotic cell; an ability to modulate entry of a mammalian cell into S phase; an ability to ubiquitinate a cell-cycle regulator; an ability to ubiquitinate a cell- or tissue-specific substrate. The cell- or tissue-specific F-box polypeptides of the present invention may also function to modulate differentiation of cells/tissue. The subject polypeptides of this invention may also be capable of modulating cell growth or proliferation by influencing the action of other cellular proteins. A cell- or tissue-specific F-box polypeptide can be a specific agonist of the function of the wild-type form of the protein, or can be a specific antagonist, such as a catalytically inactive mutant. Other biological activities of the subject cell- or tissue-specific F-box proteins are described herein, or will be reasonably apparent to those skilled in the art in light of the present disclosure.

In one embodiment, the nucleic acid of the invention encodes a polypeptide which is an agonist or antagonist of a naturally occurring vertebrate cell- or tissue-specific F-box gene product and comprises an amino acid sequence having an F-box motif (supra). Preferred cell- or tissue-specific F-box proteins are identical or homologous to the amino acid sequence represented in FIG. 5B. Preferred nucleic acids encode a polypeptide at least 60% homologous, more preferably 70% homologous and most preferably 80% homologous with an amino acid sequence shown in FIG. 5B. Nucleic acids which encode polypeptides having an activity of a cell- or tissue-specific F-box protein and having at least about 90%, more preferably at least about 95%, and most preferably at least about 98-99% homology with a sequence shown in FIG. 5A are also within the scope of the invention. Preferably, the nucleic acid is a cDNA molecule comprising at least a portion of the nucleotide sequence encoding human atrophin-1 protein shown in FIG. 5B. A preferred portion of the cDNA molecule designated in FIG. 5A includes the coding region of the molecule.

Isolated nucleic acids which differ from the nucleotide sequence shown in FIG. 5A due to degeneracy in the genetic code are also within the scope of the invention. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject cell- or tissue-specific F-box proteins will exist among mammalian cells. One skilled in the art will appreciate that these variations in one or more nucleotides (up to about 3-5% of the nucleotides) of the nucleic acids encoding a particular cell- or tissue-specific F-box protein may exist among individuals of a given species due to natural allelic variation. Any and all such nucleotide variations and resulting amino acid polymorphisms are within the scope of this invention.

The present invention pertains to nucleic acids encoding cell- or tissue-specific F-box proteins derived from a eukaryotic cell and which have amino acid sequences evolutionarily related to a cell- or tissue-specific F-box protein represented in FIG. 5B wherein “evolutionarily related to”, refers to cell- or tissue-specific F-box proteins having amino acid sequences which have arisen naturally (e.g. by allelic variance or by differential splicing), as well as mutational variants of cell- or tissue-specific F-box proteins which are derived, for example, by combinatorial mutagenesis.

Fragments of the nucleic acid encoding a biologically active portion of the subject cell- or tissue-specific F-box proteins are also within the scope of the invention. As used herein, a fragment of the nucleic acid encoding an active portion of a cell- or tissue-specific F-box protein refers to a nucleotide sequence having fewer nucleotides than the nucleotide sequence encoding the full length amino acid sequence of, for example, the cell- or tissue-specific F-box protein represented in FIG. 5B, and which encodes a polypeptide which retains at least a portion of the biological activity of the full-length protein as defined herein, or alternatively, which is functional as an antagonist of the biological activity of the full-length protein. For example, such fragments include, as appropriate to the full-length protein from which they are derived, a polypeptide containing a domain mediating the interaction of the cell- or tissue-specific F-box protein with another protein. For example, a biologically active portion of a cell- or tissue-specific F-box protein can be a portion of a cell- or tissue-specific F-box protein of the invention which is capable of interacting with a cullins protein, with a ubiquitin conjugating enzyme, with a skp1 protein, with a Rbx1 protein and/or with a substrate protein. Particularly preferred biologically active portions of vertebrate cell- or tissue-specific F-box proteins of the invention include the F box, as defined by the following consensus sequence:

ZJXZPZUZZXXZZXXXXXXXZZXZXXVXBBZXXZZXXXXZOXXZ

wherein Z is a nonpolar amino acid residue (ala, val, leu, iso, pro, phe, met, trp), X is any amino acid residue, B is a basic amino acid residue (lys, arg, his), U is an acidic amino acid residue (asp, glu), O is an aromatic amino acid residue (phe, tyr, trp), J is either serine or threonine (ser, thr), and P and V are the standard single letter representations for proline and valine, respectively (Craig and Tyers, Prog. Biophys. & Mol. Biol. 72: 299-328 (1999)); or which corresponds to from about residues 222-269 of FIG. 5B. The corresponding domains in other cell- or tissue-specific F-box protein homologs can be identified by sequence comparison with atrophin-1 as shown in FIG. 5B. Other preferred domains of cell- or tissue-specific F-box proteins include domains of the protein which mediate interaction with yet other proteins (e.g. WD40 domains or leucine rich regions (LRR domains)).

Nucleic acids within the scope of the invention may also contain linker sequences, modified restriction endonuclease sites and other sequences useful for molecular cloning, expression or purification of such recombinant polypeptides.

As indicated by the examples set out below, a nucleic acid encoding a cell- or tissue-specific F-box polypeptide may be obtained from mRNA or genomic DNA from any vertebrate organism in accordance with protocols described herein, as well as those generally known to those skilled in the art. A cDNA encoding a cell- or tissue-specific F-box polypeptide, for example, can be obtained by isolating total mRNA from a cell, e.g. a mammalian cell, e.g. a human cell. Double stranded cDNAs can then be prepared from the total mRNA, and subsequently inserted into a suitable plasmid or bacteriophage vector using any one of a number of known techniques. A gene encoding a cell- or tissue-specific F-box protein can also be cloned using established polymerase chain reaction techniques in accordance with the nucleotide sequence information provided by the invention.

Another aspect of the invention relates to the use of the isolated nucleic acid in “antisense” therapy. As used herein, antisense therapy refers to administration or in situ generation of oligonucleotide probes or their derivatives which specifically hybridize (e.g. binds) under cellular conditions with the cellular mRNA and/or genomic DNA encoding one of the subject cell- or tissue-specific F-box proteins so as to inhibit expression of that protein, e.g. by inhibiting transcription and/or translation. The binding may be by conventional base pair complementarity, or, for example, in the case of binding to DNA duplexes, through specific interactions in the major groove of the double helix. In general, antisense therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular mRNA which encodes a cell- or tissue-specific F-box protein. Alternatively, the antisense construct is an oligonucleotide probe which is generated ex vivo and which, when introduced into the cell causes inhibition of expression by hybridizing with the mRNA and/or genomic sequences encoding a cell- or tissue-specific F-box protein. Such oligonucleotide probes are preferably modified oligonucleotide which are resistant to endogenous nucleases, e.g. exonucleases and/or endonucleases, and is therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by van der Krol et al., (1988) Biotechniques 6:958-976; and Stein et al., (1988) Cancer Res 48:2659-2668.

Accordingly, the modified oligomers of the invention are useful in therapeutic, diagnostic, and research contexts. In therapeutic applications, the oligomers are utilized in a manner appropriate for antisense therapy in general. For such therapy, the oligomers of the invention can be formulated for a variety of modes of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous for injection, the oligomers of the invention can be formulated in liquid solutions, preferably in physiologically compatible buffers such as Hank's solution or Ringer's solution. In addition, the oligomers may be formulated in solid form and redissolved or suspended immediately prior to use. Lyophilized forms are also included.

Systemic administration can also be by transmucosal or transdermal means, or the compounds can be administered orally. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For oral administration, the oligomers are formulated into conventional oral administration forms such as capsules, tablets, and tonics. For topical administration, the oligomers of the invention are formulated into ointments, salves, gels, or creams as generally known in the art.

In addition to use in therapy, the oligomers of the invention may be used as diagnostic reagents to detect the presence or absence of the target DNA or RNA sequences to which they specifically bind, such as for determining the level of expression of a gene of the invention or for determining whether a gene of the invention contains a genetic lesion.

In another aspect of the invention, the subject nucleic acid is provided in an expression vector comprising a nucleotide sequence encoding a subject cell- or tissue-specific F-box polypeptide and operably linked to at least one regulatory sequence. Operably linked is intended to mean that the nucleotide sequence is linked to a regulatory sequence in a manner which allows expression of the nucleotide sequence. Regulatory sequences are art-recognized and are selected to direct expression of the polypeptide having an activity of a cell- or tissue-specific F-box protein. Accordingly, the term regulatory sequence includes promoters, enhancers and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding the cell- or tissue-specific F-box proteins of this invention. Such useful expression control sequences, include, for example, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

As will be apparent, the subject gene constructs can be used to cause expression of the subject cell- or tissue-specific F-box polypeptides in cells propagated in culture, e.g. to produce proteins or polypeptides, including fusion proteins or polypeptides, for purification.

This invention also pertains to a host cell transfected with a recombinant cell- or tissue-specific F-box gene in order to express a polypeptide having an activity of a cell- or tissue-specific F-box protein. The host cell may be any prokaryotic or eukaryotic cell. For example, a cell- or tissue-specific F-box polypeptide of the present invention may be expressed in bacterial cells such as E. coli, insect cells (baculovirus), yeast, or mammalian cells. Other suitable host cells are known to those skilled in the art.

Accordingly, the present invention further pertains to methods of producing the subject cell- or tissue-specific F-box polypeptides. For example, a host cell transfected with an expression vector encoding a cell- or tissue-specific F-box polypeptide can be cultured under appropriate conditions to allow expression of the polypeptide to occur. The polypeptide may be secreted and isolated from a mixture of cells and medium containing the polypeptide. Alternatively, the polypeptide may be retained cytoplasmically and the cells harvested, lysed and the protein isolated. A cell culture includes host cells, media and other byproducts. Suitable media for cell culture are well known in the art. The polypeptide can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the cell- or tissue-specific F-box protein. In a preferred embodiment, the cell- or tissue-specific F-box protein is a fusion protein containing a domain which facilitates its purification, such as a cell- or tissue-specific F-box-GST fusion protein.

Thus, a nucleotide sequence derived from the cloning of a cell- or tissue-specific F-box protein described in the present invention, encoding all or a selected portion of the protein, can be used to produce a recombinant form of the protein via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial cells), are standard procedures. Similar procedures, or modifications thereof, can be employed to prepare recombinant cell- or tissue-specific F-box proteins, or portions thereof, by microbial means or tissue-culture technology in accord with the subject invention.

The recombinant cell- or tissue-specific F-box protein can be produced by ligating the cloned gene, or a portion thereof, into a vector suitable for expression in either prokaryotic cells, eukaryotic cells, or both. Expression vehicles for production of a recombinant cell- or tissue-specific F-box protein include plasmids and other vectors. For instance, suitable vectors for the expression of a cell- or tissue-specific F-box protein include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al., (1983) in Experimental Manipulation of Gene Expression, ed. M. Inouye Academic Press, p. 83, incorporated by reference herein). These vectors can replicate in E. coli due the presence of the pBR322 ori, and in S. cerevisiae due to the replication determinant of the yeast 2 micron plasmid. In addition, drug resistance markers such as ampicillin can be used.

The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant cell- or tissue-specific F-box protein by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

When expression of a carboxy terminal fragment of the full-length cell- or tissue-specific F-box proteins is desired, i.e. a truncation mutant, it may be necessary to add a start codon (ATG) to the oligonucleotide fragment containing the desired sequence to be expressed. It is well known in the art that a methionine at the N-terminal position can be enzymatically cleaved by the use of the enzyme methionine aminopeptidase (MAP). MAP has been cloned from E. coli (Ben-Bassat et al., (1987) J. Bacteriol. 169:751-757) and Salmonella typhimurium and its in vitro activity has been demonstrated on recombinant proteins (Miller et al., (1987) PNAS USA 84:2718-1722). Therefore, removal of an N-terminal methionine, if desired, can be achieved either in vivo by expressing such recombinant polypeptides in a host which produces MAP (e.g., E. coli or CM89 or S. cerevisiae), or in vitro by use of purified MAP (e.g., procedure of Miller et al.).

Alternatively, the coding sequences for the polypeptide can be incorporated as a part of a fusion gene including a nucleotide sequence encoding a different polypeptide. This type of expression system can be useful under conditions where it is desirable, e.g., to produce an immunogenic fragment of the cell- or tissue-specific F-box protein. For example, the VP6 capsid protein of rotavirus can be used as an immunologic carrier protein for portions of polypeptide, either in the monomeric form or in the form of a viral particle. The nucleic acid sequences corresponding to the portion of the cell- or tissue-specific F-box protein to which antibodies are to be raised can be incorporated into a fusion gene construct which includes coding sequences for a late vaccinia virus structural protein to produce a set of recombinant viruses expressing fusion proteins comprising a portion of the protein as part of the virion. The Hepatitis B surface antigen can also be utilized in this role as well. Similarly, chimeric constructs coding for fusion proteins containing a portion of a cell- or tissue-specific F-box protein and the poliovirus capsid protein can be created to enhance immunogenicity (see, for example, EP Publication NO: 0259149; and Evans et al., (1989) Nature 339:385; Huang et al., (1988) J. Virol. 62:3855; and Schlienger et al., (1992) J. Virol. 66:2).

The Multiple Antigen Peptide system for peptide-based immunization can be utilized, wherein a desired portion of a cell- or tissue-specific F-box protein is obtained directly from organo-chemical synthesis of the peptide onto an oligomeric branching lysine core (see, for example, Posnett et al., (1988) JBC 263:1719 and Nardelli et al., (1992) J. Immunol. 148:914). Antigenic determinants of the cell- or tissue-specific F-box protein can also be expressed and presented by bacterial cells.

In addition to utilizing fusion proteins to enhance immunogenicity, it is widely appreciated that fusion proteins can also facilitate the expression of proteins. For example, the cell- or tissue-specific F-box protein of the present invention can be generated as a glutathione-S-transferase (GST) fusion proteins. Such GST fusion proteins can be used to simply purification of the cell- or tissue-specific F-box protein, such as through the use of glutathione-derivatized matrices (see, for example, Current Protocols in Molecular Biology, eds. Ausubel et al., (N.Y.: John Wiley & Sons, 1991)).

In another embodiment, a fusion gene coding for a purification leader sequence, such as a poly-(His)/enterokinase cleavage site sequence at the N-terminus of the desired portion of the recombinant protein, can allow purification of the expressed fusion protein by affinity chromatography using a Ni²⁺ metal resin. The purification leader sequence can then be subsequently removed by treatment with enterokinase to provide the purified cell- or tissue-specific F-box protein (e.g., see Hochuli et al., (1987) J. Chromatography 411:177; and Janknecht et al., PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

4. Cell- or Tissue-Specific F-Box Polypeptides

The present invention also makes available isolated and/or purified forms of the subject cell- or tissue-specific F-box polypeptides, which are isolated from, or otherwise substantially free of other intracellular proteins, especially ubiquitin conjugating enzymes, e.g. E2 enzymes, which might normally be associated with the cell- or tissue-specific F-box protein. The term “substantially free of other cellular proteins” (also referred to herein as “contaminating proteins”) is defined as encompassing, for example, cell- or tissue-specific F-box protein preparations comprising less than 20% (by dry weight) contaminating protein, and preferably comprises less than 5% contaminating protein. Functional forms of the cell- or tissue-specific F-box polypeptide can be prepared, for the first time, as purified preparations by using a cloned gene as described herein. By “purified”, it is meant, when referring to a polypeptide, that the indicated molecule is present in the substantial absence of other biological macromolecules, such as other proteins (contaminating proteins). The term “purified” as used herein preferably means at least 80% by dry weight, more preferably in the range of 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above. “Isolated” and “purified” do not encompass either natural materials in their native state or natural materials that have been separated into components (e.g., in an acrylamide gel) but not obtained either as pure (e.g. lacking contaminating proteins, or chromatography reagents such as denaturing agents and polymers, e.g. acrylamide or agarose) substances or solutions.

The subject polypeptides can also be provided in pharmaceutically acceptable carriers for formulated for a variety of modes of administration, including systemic and topical or localized administration. Techniques and formulations generally may be found in Remmington's Pharmaceutical Sciences, Meade Publishing Co., Easton, Pa. In an exemplary embodiment, the cell- or tissue-specific F-box polypeptide is provided for transmucosal or transdermal delivery. For such administration, penetrants appropriate to the barrier to be permeated are used in the formulation with the polypeptide. Such penetrants are generally known in the art, and include, for example, for transmucosal administration bile salts and fusidic acid derivatives. In addition, detergents may be used to facilitate permeation. Transmucosal administration may be through nasal sprays or using suppositories. For topical administration, the oligomers of the invention are formulated into ointments, salves, gels, or creams as generally known in the art.

Another aspect of the invention relates to polypeptides derived from the full-length cell- or tissue-specific F-box protein. Isolated peptidyl portions of the subject cell- or tissue-specific F-box protein can be obtained by screening polypeptides recombinantly produced from the corresponding fragment of the nucleic acid encoding such polypeptides. In addition, fragments can be chemically synthesized using techniques known in the art such as conventional Merrifield solid phase f-Moc or t-Boc chemistry. For example, cell- or tissue-specific F-box proteins can be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of, for example, degradation of a substrate protein, such as by microinjection assays. In an illustrative embodiment, peptidyl portions of a cell- or tissue-specific F-box protein can tested for Skp1-, cullin-, Rbx1- or substrate-binding activity, as well as inhibitory ability, by expression as, for example, thioredoxin fusion proteins, each of which contains a discrete fragment of the cell- or tissue-specific F-box protein (see, for example, U.S. Pat. Nos. 5,270,181 and 5,292,646; and PCT publication WO94/02502).

It is also possible to modify the structure of the subject cell- or tissue-specific F-box proteins for such purposes as enhancing therapeutic or prophylactic efficacy, or stability (e.g., ex vivo shelf life and resistance to proteolytic degradation in vivo). Such modified polypeptides, when designed to retain at least one activity of the naturally-occurring form of the protein, are considered functional equivalents of the cell- or tissue-specific F-box polypeptides described in more detail herein. Such modified polypeptides can be produced, for instance, by amino acid substitution, deletion, or addition.

For instance, it is reasonable to expect, for example, that an isolated replacement of a leucine with an isoleucine or valine, an aspartate with a glutamate, a threonine with a serine, or a similar replacement of an amino acid with a structurally related amino acid (i.e. conservative mutations) will not have a major effect on the biological activity of the resulting molecule. Conservative replacements are those that take place within a family of amino acids that are related in their side chains. Genetically encoded amino acids are can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 2nd ed., Ed. by L. Stryer, W. H. Freeman and Co., 1981). Whether a change in the amino acid sequence of a polypeptide results in a functional homolog can be readily determined by assessing the ability of the variant polypeptide to produce a response in cells in a fashion similar to the wild-type protein. For instance, such variant forms of a cell- or tissue-specific F-box polypeptide can be assessed, e.g., for their ability to bind to another polypeptide, e.g., Skp1, cullin, Rbx1 or a substrate. Polypeptides in which more than one replacement has taken place can readily be tested in the same manner.

This invention further contemplates a method of generating sets of combinatorial mutants of the subject cell- or tissue-specific F-box proteins, as well as truncation mutants, and is especially useful for identifying potential variant sequences (e.g. homologs) that are functional in binding to SKP1, CDC53, RBXI or a substrate protein. The purpose of screening such combinatorial libraries is to generate, for example, cell- or tissue-specific F-box homologs which can act as either agonists or antagonist, or alternatively, which possess novel activities all together. Combinatorially-derived homologs can be generated which have a selective potency relative to a naturally occurring cell- or tissue-specific F-box protein. Such proteins, when expressed from recombinant DNA constructs, can be used in gene therapy protocols.

Likewise, mutagenesis can give rise to homologs which have intracellular half-lives dramatically different than the corresponding wild-type protein. For example, the altered protein can be rendered either more stable or less stable to proteolytic degradation or other cellular process which result in destruction of, or otherwise inactivation of the cell- or tissue-specific F-box protein. Such homologs, and the genes which encode them, can be utilized to alter cell- or tissue-specific F-box protein expression by modulating the half-life of the protein. For instance, a short half-life can give rise to more transient biological effects and, when part of an inducible expression system, can allow tighter control of recombinant cell- or tissue-specific F-box protein levels within the cell. As above, such proteins, and particularly their recombinant nucleic acid constructs, can be used in gene therapy protocols.

In similar fashion, cell- or tissue-specific F-box protein homologs can be generated by the present combinatorial approach to act as antagonists, in that they are able to interfere with the ability of the corresponding wild-type protein to regulate cell ubiquitination.

In a representative embodiment of this method, the amino acid sequences for a population of cell- or tissue-specific F-box protein homologs are aligned, preferably to promote the highest homology possible. Such a population of variants can include, for example, homologs from one or more species, or homologs from the same species but which differ due to mutation. Amino acids which appear at each position of the aligned sequences are selected to create a degenerate set of combinatorial sequences. In a preferred embodiment, the combinatorial library is produced by way of a degenerate library of genes encoding a library of polypeptides which each include at least a portion of potential cell- or tissue-specific F-box protein sequences. For instance, a mixture of synthetic oligonucleotides can be enzymatically ligated into gene sequences such that the degenerate set of potential cell- or tissue-specific F-box nucleotide sequences are expressible as individual polypeptides, or alternatively, as a set of larger fusion proteins (e.g. for phage display).

There are many ways by which the library of potential homologs can be generated from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential cell- or tissue-specific F-box sequences. The synthesis of degenerate oligonucleotides is well known in the art (see for example, Narang, SA (1983) Tetrahedron 39:3; Itakura et al., (1981) Recombinant DNA, Proc. 3rd Cleveland Sympos. Macromolecules, ed. AG Walton, Amsterdam: Elsevier pp273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos. 5,223,409, 5,198,346, and 5,096,815).

Alternatively, other forms of mutagenesis can be utilized to generate a combinatorial library. For example, cell- or tissue-specific F-box protein homologs (both agonist and antagonist forms) can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is on attractive method for identifying truncated (bioactive) forms of the cell- or tissue-specific F-box proteins.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of cell- or tissue-specific F-box protein homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate sequences created by combinatorial mutagenesis techniques.

In an illustrative embodiment of a screening assay, candidate cell- or tissue-specific F-box combinatorial gene products, are displayed on the surface of a cell, and the ability of particular cells or viral particles to bind skp1, cullin, Rbx1, a substrate protein, or other binding partners via this gene product is detected in a “panning assay”. For instance, the cell- or tissue-specific F-box gene library can be cloned into the gene for a surface membrane protein of a bacterial cell (Ladner et al., WO 88/06630; Fuchs et al., (1991) Bio/Technology 9:1370-1371; and Goward et al., (1992) TIBS 18:136-140), and the resulting fusion protein detected by panning, e.g. using a fluorescently labeled molecule which binds the cell- or tissue-specific F-box protein, e.g. FITC-substrate, to score for potentially functional homologs. Cells can be visually inspected and separated under a fluorescence microscope, or, where the morphology of the cell permits, separated by a fluorescence-activated cell sorter. While the preceding description is directed to embodiments exploiting the interaction between a cell- or tissue-specific F-box polypeptide and a substrate polypeptide, it will be understood that similar embodiments can be generated using, for example, a cell- or tissue-specific F-box polypeptide displayed on the surface of a cell and examining the ability of those cell- or tissue-specific F-box-expressing cells to bind other binding partners of the cell- or tissue-specific F-box protein.

In similar fashion, the gene library can be expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at very high concentrations, a large number of phage can be screened at one time. Second, since each infectious phage displays the combinatorial gene product on its surface, if a particular phage is recovered from an affinity matrix in low yield, the phage can be amplified by another round of infection. The group of almost identical E. coli filamentous phages M13, fd, and fl are most often used in phage display libraries, as either of the phage gIII or gVIII coat proteins can be used to generate fusion proteins without disrupting the ultimate packaging of the viral particle (Ladner et al., PCT publication WO 90/02909; Garrard et al., PCT publication WO 92/09690; Marks et al., (1992) J. Biol. Chem. 267:16007-16010; Griffiths et al., (1993) EMBO J. 12:725-734; Clackson et al., (1991) Nature 352:624-628; and Barbas et al., (1992) PNAS USA 89:4457-4461).

The invention also provides for reduction of the subject cell- or tissue-specific F-box proteins to generate mimetics, e.g. peptide or non-peptide agents, which are able to mimic binding of the authentic protein to another cellular partner. Such mutagenic techniques as described above, as well as the thioredoxin system, are also particularly useful for mapping the determinants of a cell- or tissue-specific F-box protein which participate in protein-protein interactions involved in, for example, binding of the subject proteins to each other. To illustrate, the critical residues of a cell- or tissue-specific F-box protein which are involved in molecular recognition of a substrate protein can be determined and used to generate cell- or tissue-specific F-box-derived peptidomimetics which bind to the substrate protein, and by inhibiting cell- or tissue-specific F-box protein binding, act to prevent its ubiquitination. By employing, for example, scanning mutagenesis to map the amino acid residues of a cell- or tissue-specific F-box protein which are involved in binding a substrate polypeptide, peptidomimetic compounds can be generated which mimic those residues in binding to the substrate. For instance, non-hydrolyzable peptide analogs of such residues can be generated using benzodiazepine (e.g., see Freidinger et al., in Peptides: Chemistry and Biology, G. R. Marshall ed., ESCOM Publisher: Leiden, Netherlands, 1988), azepine (e.g., see Huffman et al., in Peptides: Chemistry and Biology, G. R. Marshall ed., ESCOM Publisher: Leiden, Netherlands, 1988), substituted gama lactam rings (Garvey et al., in Peptides: Chemistry and Biology, G. R. Marshall ed., ESCOM Publisher: Leiden, Netherlands, 1988), keto-methylene pseudopeptides (Ewenson et al., (1986) J. Med. Chem. 29:295; and Ewenson et al., in Peptides: Structure and Function (Proceedings of the 9th American Peptide Symposium) Pierce Chemical Co. Rockland, Ill., 1985), β-turn dipeptide cores (Nagai et al., (1985) Tetrahedron Lett 26:647; and Sato et al., (1986) J Chem Soc Perkin Trans 1:1231), and β-aminoalcohols (Gordon et al., (1985) Biochem Biophys Res Commun 126:419; and Dann et al., (1986) Biochem Biophys Res Commun 134:71).

5. Homology Searching of Nucleotide and Polypeptide Sequences

The nucleotide or amino acid sequences of the invention may be used as query sequences against databases such as GenBank, SwissProt, BLOCKS, and Pima II. These databases contain previously identified and annotated sequences that can be searched for regions of homology (similarity) using BLAST, which stands for Basic Local Alignment Search Tool (Altschul S F (1993) J Mol Evol 36:290-300; Altschul, S F et al (1990) J Mol Biol 215:403-10).

BLAST produces alignments of both nucleotide and amino acid sequences to determine sequence similarity. Because of the local nature of the alignments, BLAST is especially useful in determining exact matches or in identifying homologs which may be of prokaryotic (bacterial) or eukaryotic (animal, fungal or plant) origin. Other algorithms such as the one described in Smith, R. F. and T. F. Smith (1992; Protein Engineering 5:35-51), incorporated herein by reference, can be used when dealing with primary sequence patterns and secondary structure gap penalties. As disclosed in this application, sequences have lengths of at least 49 nucleotides and no more than 12% uncalled bases (where N is recorded rather than A, C, G, or T).

The BLAST approach, as detailed in Karlin and Altschul (1993; Proc Nat Acad Sci 90:5873-7) and incorporated herein by reference, searches matches between a query sequence and a database sequence, to evaluate the statistical significance of any matches found, and to report only those matches which satisfy the user-selected threshold of significance. Preferably the threshold is set at 10-25 for nucleotides and 3-15 for peptides.

6. Antibodies to Cell- or Tissue-Specific F-Box Polypeptides

Another aspect of the invention pertains to an antibody specifically reactive with a cell- or tissue-specific F-box protein. For example, by using peptides based on the sequence of the subject vertebrate cell- or tissue-specific F-box protein, such as atrophin-1 antisera or atrophin-1 monoclonal antibodies, can be made using standard methods. A mammal such as a mouse, a hamster or rabbit can be immunized with an immunogenic form of the peptide (e.g., an antigenic fragment which is capable of eliciting an antibody response). Techniques for conferring immunogenicity on a protein or peptide include conjugation to carriers or other techniques well known in the art. For instance, a peptidyl portion of the protein represented in FIG. 5B can be administered in the presence of adjuvant. The progress of immunization can be monitored by detection of antibody titers in plasma or serum. Standard ELISA or other immunoassays can be used with the immunogen as antigen to assess the levels of antibodies.

Following immunization, anti-cell- or tissue-specific F-box protein antisera can be obtained and, if desired, polyclonal anti-cell- or tissue-specific F-box protein antibodies isolated from the serum. To produce monoclonal antibodies, antibody producing cells (lymphocytes) can be harvested from an immunized animal and fused by standard somatic cell fusion procedures with immortalizing cells such as myeloma cells to yield hybridoma cells. Such techniques are well known in the art, an include, for example, the hybridoma technique (originally developed by Kohler and Milstein, (1975) Nature, 256: 495-497), as the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with the cell- or tissue-specific F-box proteins and the monoclonal antibodies isolated.

The term antibody as used herein is intended to include fragments thereof which are also specifically reactive with a vertebrate, e.g., mammalian cell- or tissue-specific F-box protein. Antibodies can be fragmented using conventional techniques and the fragments screened for utility in the same manner as described above for whole antibodies. For example, F(ab′)₂ fragments can be generated by treating antibody with pepsin. The resulting F(ab′)₂ fragment can be treated to reduce disulfide bridges to produce Fab′ fragments. The antibody of the present invention is further intended to include bispecific and chimeric molecules, as well as single chain (scFv) antibodies.

Particularly preferred antibodies specific for cell- or tissue-specific F-box polypeptides include trimeric antibodies and humanized antibodies, which can be prepared as described, e.g., in U.S. Pat. No. 5,585,089. Also within the scope of the invention are single chain antibodies. All of these modified forms of antibodies as well as fragments of antibodies are intended to be included in the term “antibody” and are included in the broader term “cell- or tissue-specific F-box binding protein”.

Both monoclonal and polyclonal antibodies (Ab) directed against the subject cell- or tissue-specific F-box protein, and antibody fragments such as Fab′ and F(ab′)₂, can be used to selectively block the action of individual cell- or tissue-specific F-box proteins and thereby regulate the cell-cycle, cell proliferation, differentiation and/or survival.

In one embodiment, anti-cell- or tissue-specific F-box protein antibodies are used in the immunological screening of cDNA libraries constructed in expression vectors, such as λgt11, λgt18-23, λZAP, and λORF8. Messenger libraries of this type, having coding sequences inserted in the correct reading frame and orientation, can produce fusion proteins. For instance, λgt11 will produce fusion proteins whose amino termini consist of β-galactosidase amino acid sequences and whose carboxy termini consist of a foreign polypeptide. Antigenic epitopes of a cell- or tissue-specific F-box protein, such as proteins antigenically related to the cell- or tissue-specific F-box protein as shown in FIG. 5B can then be detected with antibodies, as, for example, reacting nitrocellulose filters lifted from infected plates with an anti-cell- or tissue-specific F-box antibody. Phage, scored by this assay, can then be isolated from the infected plate. Thus, cell- or tissue-specific F-box protein homologs can be detected and cloned from other sources.

7. Transgenic Animals

Still another aspect of the invention features transgenic non-human animals which express a heterologous cell- or tissue-specific F-box gene of the present invention, or which have had one or more genomic cell- or tissue-specific F-box gene(s) disrupted in at least one of the tissue or cell-types of the animal. For instance, transgenic mice that are disrupted at their cell- or tissue-specific F-box gene locus can be generated, e.g., by homologous recombination.

In another aspect, the invention features an animal model for developmental diseases, which has a cell- or tissue-specific F-box allele which is misexpressed. For example, a mouse can be bred which has a cell- or tissue-specific F-box allele deleted, or in which all or part of one or more cell- or tissue-specific F-box exons are deleted. Such a mouse model can then be used to study disorders arising from misexpression of the cell- or tissue-specific F-box gene.

Accordingly, the present invention concerns transgenic animals which are comprised of cells (of that animal) which contain a transgene of the present invention and which preferably (though optionally) express an exogenous cell- or tissue-specific F-box protein in one or more cells in the animal. The cell- or tissue-specific F-box transgene can encode the wild-type form of the protein, or can encode homologs thereof, including both agonists and antagonists, as well as antisense constructs. In preferred embodiments, the expression of the transgene is restricted to specific subsets of cells, tissues or developmental stages utilizing, for example, cis-acting sequences that control expression in the desired pattern. In the present invention, such mosaic expression of the subject protein can be essential for many forms of lineage analysis and can additionally provide a means to assess the effects of, for example, modulation of substrate protein levels. Toward this end, tissue-specific regulatory sequences and conditional regulatory sequences can be used to control expression of the transgene in certain spatial patterns. Moreover, temporal patterns of expression can be provided by, for example, conditional recombination systems or prokaryotic transcriptional regulatory sequences.

Genetic techniques which allow for the expression of transgenes can be regulated via site-specific genetic manipulation in vivo are known to those skilled in the art. For instance, genetic systems are available which allow for the regulated expression of a recombinase that catalyzes the genetic recombination a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity. Recombinase catalyzed recombination events can be designed such that recombination of the target sequence results in either the activation or repression of expression of the subject cell- or tissue-specific F-box polypeptides. For example, excision of a target sequence which interferes with the expression of a recombinant cell- or tissue-specific F-box gene can be designed to activate expression of that gene. This interference with expression of the protein can result from a variety of mechanisms, such as spatial separation of the cell- or tissue-specific F-box gene from the promoter element or an internal stop codon. Moreover, the transgene can be made wherein the coding sequence of the gene is flanked recombinase recognition sequences and is initially transfected into cells in a 3′ to 5′ orientation with respect to the promoter element. In such an instance, inversion of the target sequence will reorient the subject gene by placing the 5′ end of the coding sequence in an orientation with respect to the promoter element which allow for promoter driven transcriptional activation.

In an illustrative embodiment, either the cre/loxP recombinase system of bacteriophage P1 (Lakso et al., (1992) PNAS USA 89:6232-6236; Orban et al., (1992) PNAS USA 89:6861-6865) or the FLP recombinase system of Saccharomyces cerevisiae (O'Gorman et al., (1991) Science 251:1351-1355; PCT publication WO 92/15694) can be used to generate in vivo site-specific genetic recombination systems. Cre recombinase catalyzes the site-specific recombination of an intervening target sequence located between loxP sequences. loxP sequences are 34 base pair nucleotide repeat sequences to which the Cre recombinase binds and are required for Cre recombinase mediated genetic recombination. The orientation of loxP sequences determines whether the intervening target sequence is excised or inverted when Cre recombinase is present (Abremski et al., (1984) J. Biol. Chem. 259:1509-1514); catalyzing the excision of the target sequence when the loxP sequences are oriented as direct repeats and catalyzes inversion of the target sequence when loxP sequences are oriented as inverted repeats.

Accordingly, genetic recombination of the target sequence is dependent on expression of the Cre recombinase. Expression of the recombinase can be regulated by promoter elements which are subject to regulatory control, e.g., tissue-specific, developmental stage-specific, inducible or repressible by externally added agents. This regulated control will result in genetic recombination of the target sequence only in cells where recombinase expression is mediated by the promoter element. Thus, the activation expression of the cell- or tissue-specific F-box gene can be regulated via regulation of recombinase expression.

Use of the cre/loxP recombinase system to regulate expression of a recombinant cell- or tissue-specific F-box protein requires the construction of a transgenic animal containing transgenes encoding both the Cre recombinase and the subject protein. Animals containing both the Cre recombinase and the recombinant cell- or tissue-specific F-box genes can be provided through the construction of “double” transgenic animals. A convenient method for providing such animals is to mate two transgenic animals each containing a transgene, e.g., the cell- or tissue-specific F-box gene and the recombinase gene.

One advantage derived from initially constructing transgenic animals containing a cell- or tissue-specific F-box transgene in a recombinase-mediated expressible format derives from the likelihood that the subject protein may be deleterious upon expression in the transgenic animal. In such an instance, a founder population, in which the subject transgene is silent in all tissues, can be propagated and maintained. Individuals of this founder population can be crossed with animals expressing the recombinase in, for example, one or more tissues. Thus, the creation of a founder population in which, for example, an antagonistic cell- or tissue-specific F-box transgene is silent will allow the study of progeny from that founder in which disruption of cell-cycle regulation in a particular tissue or at developmental stages would result in, for example, a lethal phenotype.

Similar conditional transgenes can be provided using prokaryotic promoter sequences which require prokaryotic proteins to be simultaneous expressed in order to facilitate expression of the transgene. Exemplary promoters and the corresponding transactivating prokaryotic proteins are given in U.S. Pat. No. 4,833,080. Moreover, expression of the conditional transgenes can be induced by gene therapy-like methods wherein a gene encoding the trans-activating protein, e.g. a recombinase or a prokaryotic protein, is delivered to the tissue and caused to be expressed, such as in a cell-type specific manner. By this method, the cell- or tissue-specific F-box transgene could remain silent into adulthood until “turned on” by the introduction of the transactivator.

In an exemplary embodiment, the “transgenic non-human animals” of the invention are produced by introducing transgenes into the germline of the non-human animal. Embryonal target cells at various developmental stages can be used to introduce transgenes. Different methods are used depending on the stage of development of the embryonal target cell. The zygote is the best target for micro-injection. In the mouse, the male pronucleus reaches the size of approximately 20 micrometers in diameter which allows reproducible injection of 1-2pl of DNA solution. The use of zygotes as a target for gene transfer has a major advantage in that in most cases the injected DNA will be incorporated into the host gene before the first cleavage (Brinster et al., (1985) PNAS USA 82:4438-4442). As a consequence, all cells of the transgenic non-human animal will carry the incorporated transgene. This will in general also be reflected in the efficient transmission of the transgene to offspring of the founder since 50% of the germ cells will harbor the transgene. Microinjection of zygotes is the preferred method for incorporating transgenes in practicing the invention.

Retroviral infection can also be used to introduce transgene into a non-human animal. The developing non-human embryo can be cultured in vitro to the blastocyst stage. During this time, the blastomeres can be targets for retroviral infection (Jaenich, R. (1976) PNAS USA 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al., (1985) PNAS USA 82:6927-6931; Van der Putten et al., (1985) PNAS USA 82:6148-6152). Transfection is easily and efficiently obtained by culturing the blastomeres on a monolayer of virus-producing cells (Van der Putten, supra; Stewart et al., (1987) EMBO J. 6:383-388). Alternatively, infection can be performed at a later stage. Virus or virus-producing cells can be injected into the blastocoele (Jahner et al., (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al., (1982) supra).

A third type of target cell for transgene introduction is the embryonal stem cell (ES). ES cells are obtained from pre-implantation embryos cultured in vitro and fused with embryos (Evans et al., (1981) Nature 292:154-156; Bradley et al., (1984) Nature 309:255-258; Gossler et al., (1986) PNAS USA 83: 9065-9069; and Robertson et al., (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

Methods of making knock-out or disruption transgenic animals are also generally known. See, for example, Manipulating the Mouse Embryo, (Cold Spring Harbor Laboratory Press, Cold Spring Harbor, N.Y., 1986). Recombinase dependent knockouts can also be generated, e.g. by homologous recombination to insert target sequences, such that tissue specific and/or temporal control of inactivation of a cell- or tissue-specific F-box gene can be controlled as above.

In a preferred embodiment, a transgenic animal comprising a disrupted atrophin-1 gene is provided. Preferably the animal is a livestock animal such as a cow, pig, sheep, goat, etc. Disruption of the atrophin-l gene will prevent atrohpin-1 mediated protein degradation and thus prevent muscle wasting so as to provide an animal with an increased muscle mass.

8. Detection of the Subject Cell- or Tissue-Specific F-Box Genes and Gene Products

Antibodies which are specifically immunoreactive with a cell- or tissue-specific F-box protein of the present invention can also be used in immunohistochemical staining of tissue samples in order to evaluate the abundance and pattern of expression of the protein. Anti-cell- or tissue-specific F-box protein antibodies can be used diagnostically in immuno-precipitation and immuno-blotting to detect and evaluate levels of one or more cell- or tissue-specific F-box proteins in tissue or cells isolated from a bodily fluid as part of a clinical testing procedure. Diagnostic assays using anti-cell- or tissue-specific F-box protein antibodies, can include, for example, immunoassays designed to aid in early diagnosis of a neoplastic or hyperplastic disorder, e.g. the presence of cancerous cells in the sample, e.g. to detect cells in which alterations in expression levels of cell- or tissue-specific F-box genes has occurred relative to normal cells.

In addition, nucleotide probes can be generated from the cloned sequence of the subject cell- or tissue-specific F-box proteins which allow for histological screening of intact tissue and tissue samples for the presence of a cell- or tissue-specific F-box protein encoding nucleic acids. Similar to the diagnostic uses of anti-cell- or tissue-specific F-box protein antibodies, the use of probes directed to cell- or tissue-specific F-box protein encoding mRNAs, or to genomic cell- or tissue-specific F-box gene sequences, can be used for both predictive and therapeutic evaluation of allelic mutations which might be manifest in, for example, neoplastic or hyperplastic disorders (e.g. unwanted cell growth) or unwanted differentiation events.

Used in conjunction with anti-cell- or tissue-specific F-box protein antibody immunoassays, the nucleotide probes can help facilitate the determination of the molecular basis for a developmental disorder which may involve some abnormality associated with expression (or lack thereof) of a cell- or tissue-specific F-box protein. For instance, variation in cell- or tissue-specific F-box protein synthesis can be differentiated from a mutation in the coding sequence.

In one embodiment, the present method provides a method for determining if a subject is at risk for a disorder characterized by protein degradation, aberrant cell proliferation and/or differentiation. In preferred embodiments, method can be generally characterized as comprising detecting, in a sample of cells from a vertebrate subject (preferably a human or other mammalian subject), the presence or absence of a genetic lesion characterized by at least one of (i) an alteration affecting the integrity of a cell- or tissue-specific F-box gene; or (ii) the misexpression of the cell- or tissue-specific F-box gene. To illustrate, such genetic lesions can be detected by ascertaining the existence of at least one of (i) a deletion of one or more nucleotides from a cell- or tissue-specific F-box gene, (ii) an addition of one or more nucleotides to a cell- or tissue-specific F-box gene, (iii) a substitution of one or more nucleotides of a cell- or tissue-specific F-box gene, (iv) a gross chromosomal rearrangement of a cell- or tissue-specific F-box gene, (v) a gross alteration in the level of a messenger RNA transcript of a cell- or tissue-specific F-box gene, (vii) aberrant modification of a cell- or tissue-specific F-box gene, such as of the methylation pattern of the genomic DNA, (vii) the presence of a non-wild type splicing pattern of a messenger RNA transcript of a cell- or tissue-specific F-box gene, (viii) a non-wild type level of a cell- or tissue-specific F-box protein, and (ix) inappropriate post-translational modification of a cell- or tissue-specific F-box protein. As set out below, the present invention provides a large number of assay techniques for detecting lesions in an cell- or tissue-specific F-box gene, and importantly, provides the ability to discern between different molecular causes underlying cell- or tissue-specific F-box dependent aberrant cell growth, proliferation, differentiation, and/or protein degredation.

In an exemplary embodiment, there is provided a nucleic acid composition comprising a (purified) oligonucleotide probe including a region of nucleotide sequence which is capable of hybridizing to a sense or antisense sequence of an cell- or tissue-specific F-box gene, such as represented by the sequence shown in FIG. 5A or naturally occurring mutants thereof, or 5′ or 3′ flanking sequences or intronic sequences naturally associated with the subject cell- or tissue-specific F-box genes or naturally occurring mutants thereof. The nucleic acid of a cell is rendered accessible for hybridization, the probe is exposed to nucleic acid of the sample, and the hybridization of the probe to the sample nucleic acid is detected. Such techniques can be used to detect lesions at either the genomic or mRNA level, including deletions, substitutions, etc., as well as to determine mRNA transcript levels.

In certain embodiments, detection of the lesion comprises utilizing the probe/primer in a polymerase chain reaction (PCR) (see, e.g. U.S. Pat. Nos. 4,683,195 and 4,683,202), such as anchor PCR or RACE PCR, or, alternatively, in a ligation chain reaction (LCR) (see, e.g., Landegran et al., (1988) Science 241:1077-1080; and Nakazawa et al., (1944) PNAS USA 91:360-364), the later of which can be particularly useful for detecting point mutations in the cell- or tissue-specific F-box gene. In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize to a cell- or tissue-specific F-box gene under conditions such that hybridization and amplification of the cell- or tissue-specific F-box gene (if present) occurs, and (iv) detecting the presence or absence of an amplification product, or detecting the size of the amplification product and comparing the length to a control sample.

In yet another exemplary embodiment, aberrant methylation patterns of a cell- or tissue-specific F-box gene can be detected by digesting genomic DNA from a patient sample with one or more restriction endonucleases that are sensitive to methylation and for which recognition sites exist in the cell- or tissue-specific F-box gene (including in the flanking and intronic sequences). See, for example, Buiting et al., (1994) Human Mol Genet 3:893-895. Digested DNA is separated by gel electrophoresis, and hybridized with probes derived from, for example, genomic or cDNA sequences. The methylation status of the cell- or tissue-specific F-box gene can be determined by comparison of the restriction pattern generated from the sample DNA with that for a standard of known methylation.

In still another embodiment, a diagnostic assay is provided which detects the ability of an cell- or tissue-specific F-box gene product, e.g., isolated from a biopsied cell, to bind to other cellular proteins. For instance, it will be desirable to detect cell- or tissue-specific F-box mutants which bind with higher or lower binding affinity for SKP1, for CDC53, for RBX1, for a ubiquitin conjugating enzyme, or for a substrate protein. Such mutants may arise, for example, from fine mutations, e.g., point mutants, which may be impractical to detect by the diagnostic DNA sequencing techniques or by the immunoassays described above. The present invention accordingly further contemplates diagnostic screening assays which generally comprise cloning one or more cell- or tissue-specific F-box genes from the sample cells, and expressing the cloned genes under conditions which permit detection of an interaction between that recombinant gene product and a substrate protein. As will be apparent from the description of the various drug screening assays set forth below, a wide variety of techniques can be used to determine the ability of a cell- or tissue-specific F-box protein to bind to other cellular components.

For example, the subject method can comprise the steps of: (i) ascertaining the level of a cell- or tissue-specific F-box protein, a cell- or tissue-specific F-box protein transcript and/or a cell- or tissue-specific F-box protein activity in a sample of cells from the patient; and (ii) evaluating, from such levels in the sample cells compared to normal cells, the aggressiveness and/or prospective rate of recurrence of a disorder marked by aberrant hyperproliferation. As will be understood by those skilled in the art, the method of the present invention can be carried out using any of a large number of assay techniques for detecting the cell- or tissue-specific F-box protein and/or its activity, and importantly, provides the ability to discern between different molecular causes underlying aberrant cell growth, proliferation, differentiation, and/or protein degradation.

9. Gene Therapy

The invention provides methods for modulating ubiquitination and subsequent degradation of substrate proteins. Accordingly, the invention provides methods for modulating cell proliferation, differentiation and/or survival, which can be used, for example. to treat diseases or conditions associated with aberrant protein degradation, cell proliferation, differentiation and/or survival. According to the methods of the invention, a cell- or tissue-specific F-box protein therapeutic is administered to a subject having a disease associated with aberrant protein degradation, cell proliferation, differentiation and/or cell survival.

There are a wide variety of pathological cell proliferative conditions for which the cell- or tissue-specific F-box gene constructs, cell- or tissue-specific F-box mimetics and cell- or tissue-specific F-box antagonists, of the present invention can provide therapeutic benefits, with the general strategy being the modulation of protein degradation in a specific cell- or tissue-type. For instance, the gene constructs of the present invention can be used as a part of a gene therapy protocol, such as to reconstitute the function of a cell- or tissue-specific F-box protein, e.g. in a cell in which the protein is misexpressed or in which signal transduction pathways upstream of a cell- or tissue-specific F-box protein are dysfunctional, or to inhibit the function of the wild-type protein, e.g. by delivery of a dominant negative mutant.

To illustrate, cell types which exhibit pathological or abnormal growth presumably dependent at least in part on a function (or dysfunction) of a cell- or tissue-specific F-box protein include various disease associated with aberrant protein degradation including muscle wasting and cachexia associated with a variety of cancers and leukemias.

It will also be apparent that, by transient use of gene therapy constructs of the subject cell- or tissue-specific F-box proteins (e.g. agonist and antagonist forms) or antisense nucleic acids, in vivo reformation of tissue can be accomplished, e.g. in the development and maintenance of organs. By controlling protein degradation in a specific cell- or tissue-type, the subject gene constructs can be used, e.g. to prevent or treat muscle wasting or cachexia, to reform injured tissue, or to improve grafting and morphology of transplanted tissue. For instance, cell- or tissue-specific F-box agonists and antagonists can be employed therapeutically to regulate organs after physical, chemical or pathological insult. For example, gene therapy can be utilized in liver repair subsequent to a partial hepatectomy, or to promote regeneration of lung tissue in the treatment of emphysema.

In one aspect of the invention, expression constructs of the subject cell- or tissue-specific F-box proteins, or for generating antisense molecules, may be administered in any biologically effective carrier, e.g. any formulation or composition capable of effectively transfecting cells in vivo with a recombinant cell- or tissue-specific F-box gene. Approaches include insertion of the subject gene in viral vectors including recombinant retroviruses, adenovirus, adeno-associated virus, and herpes simplex virus-1, or recombinant bacterial or eukaryotic plasmids. Viral vectors can be used to transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO₄ precipitation carried out in vivo. It will be appreciated that because transduction of appropriate target cells represents the critical first step in gene therapy, choice of the particular gene delivery system will depend on such factors as the phenotype of the intended target and the route of administration, e.g. locally or systemically.

A preferred approach for in vivo introduction of nucleic acid encoding one of the subject proteins into a cell is by use of a viral vector containing nucleic acid, e.g. a cDNA, encoding the gene product. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors are generally understood to be the recombinant gene delivery system of choice for the transfer of exogenous genes in vivo, particularly into humans. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid encoding a cell- or tissue-specific F-box polypeptide, rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al., (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include ψCrip, ψCre, ψ2 and ψAm. Retroviruses have been used to introduce a variety of genes into many different cell types, including neural cells, epithelial cells, endothelial cells, lymphocytes, myoblasts, hepatocytes, bone marrow cells, in vitro and/or in vivo (see for example Eglitis et al., (1985) Science 230:1395-1398; Danos and Mulligan, (1988) PNAS USA 85:6460-6464; Wilson et al., (1988) PNAS USA 85:3014-3018; Armentano et al., (1990) PNAS USA 87:6141-6145; Huber et al., (1991) PNAS USA 88:8039-8043; Ferry et al., (1991) PNAS USA 88:8377-8381; Chowdhury et al., (1991) Science 254:1802-1805; van Beusechem et al., (1992) PNAS USA 89:7640-7644; Kay et al., (1992) Human Gene Therapy 3:641-647; Dai et al., (1992) PNAS USA 89:10892-10895; Hwu et al., (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications WO93/25234, WO94/06920, and WO94/11524). For instance, strategies for the modification of the infection spectrum of retroviral vectors include: coupling antibodies specific for cell surface antigens to the viral env protein (Roux et al., (1989) PNAS USA 86:9079-9083; Julan et al., (1992) J. Gen Virol 73:3251-3255; and Goud et al., (1983) Virology 163:251-254); or coupling cell surface ligands to the viral env proteins (Neda et al., (1991) J. Biol. Chem. 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, and can also be used to convert an ecotropic vector in to an amphotropic vector.

Another viral gene delivery system useful in the present invention utilizes adenovirus-derived vectors. The genome of an adenovirus can be manipulated such that it encodes a gene product of interest, but is inactivate in terms of its ability to replicate in a normal lytic viral life cycle (see, for example, Berkner et al., (1988) BioTechniques 6:616; Rosenfeld et al., (1991) Science 252:431-434; and Rosenfeld et al., (1992) Cell 68:143-155). Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses can be advantageous in certain circumstances in that they are not capable of infecting nondividing cells and can be used to infect a wide variety of cell types, including airway epithelium (Rosenfeld et al., (1992) cited supra), endothelial cells (Lemarchand et al., (1992) PNAS USA 89:6482-6486), hepatocytes (Herz and Gerard, (1993) PNAS USA 90:2812-2816) and muscle cells (Quantin et al., (1992) PNAS USA 89:2581-2584). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al., (1979) Cell 16:683; Berkner et al., supra; and Graham et al., in Methods in Molecular Biology, E. J. Murray, Ed. (Humana, Clifton, N.J., 1991) vol. 7. pp. 109-127). Expression of the inserted cell- or tissue-specific F-box gene can be under control of, for example, the E1A promoter, the major late promoter (MLP) and associated leader sequences, the viral E3 promoter, or exogenously added promoter sequences.

Yet another viral vector system useful for delivery of the subject cell- or tissue-specific F-box genes is the adeno-associated virus (AAV). Adeno-associated virus is a naturally occurring defective virus that requires another virus, such as an adenovirus or a herpes virus, as a helper virus for efficient replication and a productive life cycle. (For a review, see Muzyczka et al., Curr. Topics in Micro. and Immunol. (1992) 158:97-129). It is also one of the few viruses that may integrate its DNA into non-dividing cells, and exhibits a high frequency of stable integration (see for example Flotte et al., (1992) Am. J. Respir. Cell. Mol. Biol. 7:349-356; Samulski et al., (1989) J. Virol. 63:3822-3828; and McLaughlin et al., (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al., (1985) Mol. Cell. Biol. 5:3251-3260 can be used to introduce DNA into cells. A variety of nucleic acids have been introduced into different cell types using AAV vectors (see for example Hermonat et al., (1984) PNAS USA 81:6466-6470; Tratschin et al., (1985) Mol. Cell. Biol. 4:2072-2081; Wondisford et al., (1988) Mol. Endocrinol. 2:32-39; Tratschin et al., (1984) J. Virol. 51:611-619; and Flotte et al., (1993) J. Biol. Chem. 268:3781-3790).

Other viral vector systems that may have application in gene therapy have been derived from herpes virus, vaccinia virus, and several RNA viruses. In particular, herpes virus vectors may provide a unique strategy for persistence of the recombinant cell- or tissue-specific F-box gene in cells of the central nervous system and ocular tissue (Pepose et al., (1994) Invest Ophthalmol Vis Sci 35:2662-2666)

In addition to viral transfer methods, such as those illustrated above, non-viral methods can also be employed to cause expression of a cell- or tissue-specific F-box protein in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject cell- or tissue-specific F-box gene by the targeted cell. Exemplary gene delivery systems of this type include liposomal derived systems, poly-lysine conjugates, and artificial viral envelopes.

In a representative embodiment, a gene encoding a cell- or tissue-specific F-box polypeptide can be entrapped in liposomes bearing positive charges on their surface (e.g., lipofectins) and (optionally) which are tagged with antibodies against cell surface antigens of the target tissue (Mizuno et al., (1992) No Shinkei Geka 20:547-551; PCT publication WO91/06309; Japanese patent application 1047381; and European patent publication EP-A-43075). For example, lipofection of neuroglioma cells can be carried out using liposomes tagged with monoclonal antibodies against glioma-associated antigen (Mizuno et al., (1992) Neurol. Med. Chir. 32:873-876).

In yet another illustrative embodiment, the gene delivery system comprises an antibody or cell surface ligand which is cross-linked with a gene binding agent such as poly-lysine (see, for example, PCT publications WO93/04701, WO92/22635, WO92/20316, WO92/19749, and WO92/06180). For example, the subject cell- or tissue-specific F-box gene construct can be used to transfect specific cells in vivo using a soluble polynucleotide carrier comprising an antibody conjugated to a polycation, e.g. poly-lysine (see U.S. Pat. No. 5,166,320). It will also be appreciated that effective delivery of the subject nucleic acid constructs via -mediated endocytosis can be improved using agents which enhance escape of the gene from the endosomal structures. For instance, whole adenovirus or fusogenic peptides of the influenza HA gene product can be used as part of the delivery system to induce efficient disruption of DNA-containing endosomes (Mulligan et al., (1993) Science 260-926; Wagner et al., (1992) PNAS USA 89:7934; and Christiano et al., (1993) PNAS USA 90:2122).

In clinical settings, the gene delivery systems can be introduced into a patient by any of a number of methods, each of which is familiar in the art. For instance, a pharmaceutical preparation of the gene delivery system can be introduced systemically, e.g. by intravenous injection, and specific transduction of the construct in the target cells occurs predominantly from specificity of transfection provided by the gene delivery vehicle, cell-type or tissue-type expression due to the transcriptional regulatory sequences controlling expression of the gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al., (1994) PNAS USA 91: 3054-3057).

10. Drug Screening Assays

The present invention also provides assays for identifying drugs which are either agonists or antagonists of the normal cellular function of the subject cell- or tissue-specific F-box proteins, or of the role of those proteins in the pathogenesis of normal or abnormal protein degredation and disorders related thereto (e.g. muscle wasting and cachexia), as mediated by, for example, the ubiquitination of substrate proteins by a cell- or tissue-specific F-box-dependent process. In one embodiment, the assay evaluates the ability of a compound to modulate binding and/or ubiquitinylation of a cellular or viral substrate by a cell- or tissue-specific F-box ligase. In other embodiments, the assay merely detects agents which inhibit interaction of one of the subject cell- or tissue-specific F-box proteins with SKP1, CDC53, RBX1 or a substrate protein. Such modulators can be used, for example, in the treatment of proliferative and/or differentiative disorders, and to modulate apoptosis, and to modulate protein degredation in a specific cell or tissue type.

A variety of assay formats will suffice and, in light of the present disclosure, those not expressly described herein will nevertheless be comprehended by one of ordinary skill in the art. Assay formats which approximate the ubiquitination of target polypeptides as mediated by E3 complexes can be generated in many different forms, and include assays based on cell-free systems, e.g. purified proteins or cell lysates, as well as cell-based assays which utilize intact cells. Simple binding assays can also be used to detect agents which, by disrupting the binding of an E2 to a cell- or tissue-specific F-box protein or complex, or the binding of a cell- or tissue-specific F-box protein or complex to a substrate, can inhibit cell- or tissue-specific F-box-dependent ubiquitination. Agents to be tested for their ability to act as cell- or tissue-specific F-box inhibitors can be produced, for example, by bacteria, yeast or other organisms (e.g. natural products), produced chemically (e.g. small molecules, including peptidomimetics), or produced recombinantly. In a preferred embodiment, the test agent is a small organic molecule, e.g., other than a peptide or oligonucleotide, having a molecular weight of less than about 2,000 daltons.

In many drug screening programs which test libraries of compounds and natural extracts, high throughput assays are desirable in order to maximize the number of compounds surveyed in a given period of time. Assays of the present invention which are performed in cell-free systems, such as may be derived with purified or semi-purified proteins or with lysates, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or changes in enzymatic properties of the molecular target. Accordingly, potential modifiers, e.g., activators or inhibitors of cell- or tissue-specific F-box-dependent ubiquitination of a polypeptide substrate can be detected in a cell-free assay generated by constitution of a functional ubiquitin conjugating system in a cell lysate, such as generated by charging a ubiquitin-depleted reticulocyte lysate (Hershko et al., (1983) J Biol Chem 258:8206-8214) with one or more of an E1 enzyme, an E2 enzyme, skp1, cullins, Rbx1, a cell- or tissue-specific F-box protein, ubiquitin, and/or a substrate for cell- or tissue-specific F-box protein-dependent ubiquitination. In an alternate format, the assay can be derived as a reconstituted protein mixture which, as described below, offers a number of benefits over lysate-based assays.

In one aspect, the present invention provides assays that can be used to screen for drugs which modulate the conjugation of ubiquitin to a substrate of a cell- or tissue-specific F-box protein. For instance, the drug screening assays of the present invention can be designed to detect agents which disrupt binding of a cell- or tissue-specific F-box protein (such as atrophin-1), to a substrate protein. In other embodiments, the subject assays will identify inhibitors of the enzymatic activity of the cell- or tissue-specific F-box protein, e.g., which inhibitors prevent transfer of ubiquitin from the SCF complex to a substrate protein, or which inhibit the transfer of ubiquitin from an E2 enzyme, such as UBC2 or UBC3, to a cell- or tissue-specific F-box protein amino acid side chain. In a preferred embodiment, the agent is a mechanism based inhibitor which chemically alters the enzyme and which is a specific inhibitor of that enzyme, e.g. has an inhibition constant 10-fold, 100-fold, or more preferably, 1000-fold different for other human E3 ligases.

In many embodiments of the subject assay which utilize a ubiquitin-conjugating system, the level of ubiquitination of a substrate polypeptide brought about by the ubiquitin-conjugating system is measured in the presence and absence of a candidate agent, and a decrease in the level of ubiquitin conjugation is indicative of an inhibitory activity for the candidate agent. As described below, the level of ubiquitination of the substrate polypeptide can be measured by determining the actual concentration of substrate:ubiquitin conjugates formed; or inferred by detecting some other quality of the subject substrate polypeptide affected by ubiquitination, including the proteolytic degradation of the protein. A statistically significant decrease in ubiquitination of the substrate polypeptide in the presence of the test compound is indicative of the test compound being an inhibitor of cell- or tissue-specific F-box protein-dependent ubiquitin conjugation of a substrate protein.

In preferred in vitro embodiments of the present assay, the ubiquitin-conjugating system comprises a reconstituted protein mixture of at least semi-purified proteins. By semi-purified, it is meant that the proteins utilized in the reconstituted mixture have been previously separated from other cellular or viral proteins. For instance, in contrast to cell lysates, the proteins involved in conjugation of ubiquitin to a substrate polypeptide, together with the substrate polypeptide, are present in the mixture to at least 50% purity relative to all other proteins in the mixture, and more preferably are present at 90-95% purity. In certain embodiments of the subject method, the reconstituted protein mixture is derived by mixing highly purified proteins such that the reconstituted mixture substantially lacks other proteins (such as of cellular or viral origin) which might interfere with or otherwise alter the ability to measure specific ubiquitination or ubiquitin-mediated degradation of the target substrate polypeptide.

With respect to measuring ubiquitination, the purified protein mixture can substantially lack any proteolytic activity which would degrade the substrate polypeptide and/or components of the ubiquitin conjugating system. For instance, the reconstituted system can be generated to have less than 10% of the proteolytic activity associated with a typical lysate, and preferably no more than 5%, and most preferably less than 2%. Alternatively, the mixture can be generated to include, either from the onset of ubiquitination or from some point after ubiquitin conjugation of the substrate polypeptide, a ubiquitin-dependent proteolytic activity, such as a purified proteosome complex, that is present in the mixture in discrete, measured amounts.

In the subject method, ubiquitin conjugating systems derived from purified proteins can hold a number of significant advantages over cell lysate or wheat germ extract based assays (collectively referred to hereinafter as “lysates”). Unlike the reconstituted protein system, the synthesis and destruction of the substrate polypeptide cannot be readily controlled for in lysate-based assays. Without knowledge of particular kinetic parameters for Ub-independent and Ub-dependent degradation of the substrate polypeptide in the lysate, discerning between the two pathways can be extremely difficult. Measuring these parameters, if at all possible, is further made tedious by the fact that cell lysates tend to be inconsistent from batch to batch, with potentially significant variation between preparations. Evaluation of a potential inhibitor using a lysate system is also complicated in those circumstances where the lysate is charged with mRNA encoding the substrate polypeptide, as such lysates may continue to synthesize the protein during the assay, and will do so at unpredictable rates.

Using similar considerations, knowledge of the concentration of each component of the ubiquitin conjugation pathway can be required for each lysate batch, along with the degradative kinetic data, in order to determine the necessary time course and calculate the sensitivity of experiments performed from one lysate preparation to the next.

Furthermore, the lysate system can be unsatisfactory where the substrate polypeptide itself has a relatively short half-life, especially if due to degradative processes other than the ubiquitin-mediated pathway to which an inhibitor is sought.

In one embodiment, the use of reconstituted protein mixtures allows more careful control of the reaction conditions in the ubiquitin-conjugating system. Moreover, the system can be derived to favor discovery of inhibitors of particular steps of the ubiquitination process. For instance, a reconstituted protein assay can be generated which does not facilitate degradation of the ubiquitinated substrate polypeptide. The level of ubiquitin conjugated substrate polypeptide can easily be measured directly in such a system, both in the presence and absence of a candidate agent, thereby enhancing the ability to detect an inhibitor of cell- or tissue-specific F-box protein-dependent ubiquitination. Alternatively, the Ub-conjugating system can be allowed to develop a steady state level of substrate:Ub conjugates in the absence of a proteolytic activity, but then shifted to a degradative system by addition of purified Ub-dependent proteases. Such degradative systems would be amenable to identifying proteosome inhibitors.

The purified protein mixture includes a purified preparation of the substrate polypeptide and the cell- or tissue-specific F-box protein under conditions which drive the conjugation of the two molecules. For instance, the mixture can include ubiquitin, a ubiquitin-activating enzyme (E1), a ubiquitin-conjugating enzyme (E2) such as UBC2 or UBC3, Skp1, cullin, Rbx1, and a nucleotide triphosphate (e.g. ATP). Alternatively, the E1 enzyme, the ubiquitin, and the nucleotide triphosphate can be substituted in the system with a pre-activated ubiquitin in the form of an E1::Ub or E2::Ub conjugate. Likewise, a pre-activated ubiquitin can instead comprise a cell- or tissue-specific F-box protein::Ub conjugate which can directly transfer the pre-activated ubiquitin to the substrate polypeptide.

Ubiquitination of the target substrate polypeptide via an in vitro ubiquitin-conjugating system, in the presence and absence of a candidate inhibitor, can be accomplished in any vessel suitable for containing the reactants. Examples include microtitre plates, test tubes, and micro-centrifuge tubes. In certain embodiments of the present assay, the in vitro assay system is generated to lack the ability to degrade the ubiquitinated substrate polypeptide. In such an embodiments, a wide range of detection means can be practiced to score for the presence of the ubiquitinated protein.

In one embodiment of the present assay, the products of a non-degradative ubiquitin-conjugating system are separated by gel electrophoresis, and the level of ubiquitinated substrate polypeptide assessed, using standard electrophoresis protocols, by measuring an increase in molecular weight of the substrate polypeptide that corresponds to the addition of one or more ubiquitin chains. For example, one or both of the substrate polypeptide and ubiquitin can be labeled with a radioisotope such as ³⁵S, ¹⁴C, or ³H, and the isotopically labeled protein bands quantified by autoradiographic techniques. Standardization of the assay samples can be accomplished, for instance, by adding known quantities of labeled proteins which are not themselves subject to ubiquitination or degradation under the conditions which the assay is performed. Similarly, other means of detecting electrophoretically separated proteins can be employed to quantify the level of ubiquitination of the substrate polypeptide, including immunoblot analysis using antibodies specific for either the substrate polypeptide or ubiquitin, or derivatives thereof. As described below, the antibody can be replaced with another molecule able to bind one of either the substrate polypeptide or ubiquitin. By way of illustration, one embodiment of the present assay comprises the use of biotinylated ubiquitin in the conjugating system. The biotin label is detected in a gel during a subsequent detection step by contacting the electrophoretic products (or a blot thereof) with a streptavidin-conjugated label, such as a streptavidin linked fluorochrome or enzyme, which can be readily detected by conventional techniques. Moreover, where a reconstituted protein mixture is used (rather than a lysate) as the conjugating system, it may be possible to simply detect the substrate polypeptide and ubiquitin conjugates thereof in the gel by standard staining protocols, including coomassie blue and silver staining.

In another embodiment, an immunoassay or similar binding assay, is used to detect and quantify the level of ubiquitinated substrate polypeptide produced in the ubiquitin-conjugating system. Many different immunoassay techniques are amenable for such use and can be employed to detect and quantitate the substrate::Ub conjugates. For example, the wells of a microtitre plate (or other suitable solid phase) can be coated with an antibody which specifically binds one of either the substrate polypeptide or ubiquitin. After incubation of the ubiquitin-conjugated system with and without the candidate agent, the products are contacted with the matrix bound antibody, unbound material removed by washing, and ubiquitin conjugates of the substrate polypeptide specifically detected. To illustrate, if an antibody which binds the substrate polypeptide is used to sequester the polypeptide on the matrix, then a detectable anti-ubiquitin antibody can be used to score for the presence of ubiquitinated substrate polypeptide on the matrix.

However, the use of antibodies in these binding assays is merely illustrative of binding molecules in general, and that the antibodies are readily substituted in the assay with any suitable molecule that can specifically detect one of either the substrate polypeptide or the ubiquitin. As described below, a biotin-derivative of ubiquitin can be used, and streptavidin (or avidin) employed to bind the biotinylated ubiquitin. In an illustrative embodiment, wells of a microtitre plate are coated with streptavidin and contacted with the developed ubiquitin-conjugating system under conditions wherein the biotinylated ubiquitin binds to and is sequestered in the wells. Unbound material is washed from the wells, and the level of substrate polypeptide (bound to the matrix via a conjugated ubiquitin moiety) is detected in each well. Alternatively, the microtitre plate wells can be coated with an antibody (or other binding molecule) which binds and sequesters the substrate polypeptide on the solid support, and detection of ubiquitinated conjugates of the matrix-bound substrate polypeptide are subsequently carried out using a detectable streptavidin derivative, such as an alkaline phosphatase/streptavidin complex.

In similar fashion, epitope-tagged ubiquitin, such as myc-ub (see Ellison et al. (1991) J. Biol. Chem. 266:21150-21157; ubiquitin which includes a 10-residue sequence encoding a protein of c-myc) can be used in conjunction with antibodies to the epitope tag. A major advantage of using such an epitope-tagged ubiquitin approach for detecting Ub:protein conjugates is the ability of an N-terminal tag sequences to inhibit ubiquitin-mediated proteolysis of the conjugated substrate polypeptide.

Other ubiquitin derivatives include detectable labels which do not interfere greatly with the conjugation of ubiquitin to the substrate polypeptide. Such detectable labels can include fluorescently-labeled (e.g. FITC) or enzymatically-labeled ubiquitin fusion proteins. These derivatives can be produced by chemical cross-linking, or, where the label is a protein, by generation of a fusion protein. Several labeled ubiquitin derivatives are commercially available.

Likewise, other binding molecules can be employed in place of the antibodies that bind the substrate polypeptide. For example, the substrate polypeptide can be generated as a glutathione-S-transferase (GST) fusion protein. As a practical matter, such GST fusion protein can enable easy purification of the substrate polypeptide in the preparation of components of the ubiquitin-conjugating system (see, for example, Current Protocols in Molecular Biology, eds. Ausubel et al. (NY: John Wiley & Sons, 1991); Smith et al. (1988) Gene 67:31; and Kaelin et al. (1992) Cell 70:351) Moreover, glutathione derivatized matrices (e.g. glutathione-sepharose or glutathione-coated microtitre plates) can be used to sequester free and ubiquitinated forms of the substrate polypeptide from the ubiquitin-conjugating system, and the level of ubiquitin immobilized can be measured as described. Likewise, where the matrix is generated to bind ubiquitin, the level of sequestered GST-substrate polypeptide can be detected using agents which bind to the GST moiety (such as anti-GST antibodies), or, alternatively, using agents which are enzymatically acted upon by GST to produce detectable products (e.g. 1-chloro-2,4-dinitrobenzene; Habig et al. (1974) J Biol Chem 249:7130). Similarly, other fusion proteins involving the substrate polypeptide and an enzymatic activity are contemplated by the present method. For example, fusion proteins containing β-galactosidase, green fluorescent protein or luciferase, to name but a few, can be employed as labels to determine the amount of substrate polypeptide sequestered on a matrix by virtue of a conjugated ubiquitin chain.

Moreover, such enzyme/substrate fusion proteins can be used to detect and quantitate ubiquitinated substrate polypeptide in a heterogeneous assay, that is one which does not require separation of the components of the conjugating system. For example, ubiquitin conjugating systems can be generated to have a ubiquitin-dependent protease which degrades the substrate fusion protein. The enzymatic activity of the fusion protein provides a detectable signal, in the presence of substrate, for measuring the level of the substrate ubiquitination. Similarly, in a non-degradative conjugating system, ubiquitination of the substrate portion of the fusion protein can allosterically influence the enzymatic activity associated with the fusion the protein and thereby provides a means for monitoring the level of ubiquitin conjugation.

In binding assay-type detection steps set out above, the choice of which of either the substrate polypeptide or ubiquitin should be specifically sequestered on the matrix will depend on a number of factors, including the relative abundance of both components in the conjugating system. For instance, where the reaction conditions of the ubiquitin conjugating system provide ubiquitin at a concentration far in excess of the level of the substrate polypeptide, (e.g., one order of magnitude or greater) sequestering the ubiquitin and detecting the amount of substrate polypeptide bound with the ubiquitin can provide less dynamic range to the detection step of the present method than the converse embodiment of sequestering the substrate polypeptide and detecting ubiquitin conjugates from the total substrate pool bound to the matrix. That is, where ubiquitin is provided in great excess relative to the substrate polypeptide, the percentage of ubiquitin conjugated substrate in the total ubiquitin bound to the matrix can be small enough that any diminishment in ubiquitination caused by an inhibitor can be made difficult to detect by the fact that, for example, the statistical error of the system (e.g. the noise) can be a significant portion of the measured change in concentration of bound substrate polypeptide. Furthermore, it is clear that manipulating the reaction conditions and reactant concentrations in the ubiquitin-conjugating system can be carried out to provide, at the detection step, greater sensitivity by ensuring that a strong ubiquitinated protein signal exists in the absence of any inhibitor.

Furthermore, drug screening assays can be generated which do not measure ubiquitination per se, but rather detect inhibitory agents on the basis of their ability to interfere with binding of the cell- or tissue-specific F-box polypeptides with a substrate polypeptide. In an exemplary binding assay, the compound of interest is contacted with a mixture generated from a cell- or tissue-specific F-box polypeptide and a substrate polypeptide. Detection and quantification of cell- or tissue-specific F-box protein:substrate complexes provides a means for determining the compound's efficacy at inhibiting (or potentiating) complex formation between the two polypeptides. The efficacy of the compound can be assessed by generating dose response curves from data obtained using various concentrations of the test compound. Moreover, a control assay can also be performed to provide a baseline for comparison. In the control assay, the formation of complexes is quantitated in the absence of the test compound. In certain embodiments, the binding assay can be carried out under conditions wherein ubiquitination of the substrate does not occur, e.g., by the use of reaction mixtures lacking Ub or generated with ubiquitination-defective cullins protein (e.g. mutated active site) or substrate protein (e.g., lacking ubiquitin substrate lysine residues).

Complex formation between the cell- or tissue-specific F-box polypeptide and substrate polypeptides may be detected by a variety of techniques, many of which are effectively described above. For instance, modulation in the formation of complexes can be quantitated using, for example, detectably labelled proteins (e.g. radiolabelled, fluorescently labelled, or enzymatically labelled), by immunoassay, or by chromatographic detection.

Typically, it will be desirable to immobilize either one of the polypeptides to facilitate separation of complexes from uncomplexed forms of one of the proteins, as well as to accommodate automation of the assay. In an illustrative embodiment, a fusion protein can be provided which adds a domain that permits the protein to be bound to an insoluble matrix. For example, GST-cell- or tissue-specific F-box protein fusions can be adsorbed onto glutathione sepharose beads (Sigma Chemical, St. Louis, Mo.) or glutathione derivatized microtitre plates, which are then combined with a substrate polypeptide, e.g. an ³⁵S-labeled polypeptide, and the test compound and incubated under conditions conducive to complex formation. Following incubation, the beads are washed to remove any unbound substrate polypeptide, and the matrix bead-bound radiolabel determined directly (e.g. beads placed in scintillant), or in the supernatant after the complexes are dissociated, e.g. when a microtitre plate is used. Alternatively, after washing away unbound protein, the complexes can be dissociated from the matrix, separated by SDS-PAGE gel, and the level of substrate polypeptide found in the matrix-bound fraction quantitated from the gel using standard electrophoretic techniques.

In yet another embodiment, the cell- or tissue-specific F-box polypeptide and substrate polypeptides can be used to generate an interaction trap assay (see also, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696), for subsequently detecting agents which disrupt binding of the proteins to one and other.

In particular, the method makes use of chimeric genes which express hybrid proteins. To illustrate, a first hybrid gene comprises the coding sequence for a DNA-binding domain of a transcriptional activator can be fused in frame to the coding sequence for a “bait” protein, e.g., a cell- or tissue-specific F-box polypeptide of sufficient length to bind to a substrate polypeptide. The second hybrid protein encodes a transcriptional activation domain fused in frame to a gene encoding a “fish” protein, e.g., a substrate polypeptide of sufficient length to interact with the substrate polypeptide portion of the bait fusion protein. If the bait and fish proteins are able to interact, e.g., form a cell- or tissue-specific F-box protein/substrate complex, they bring into close proximity the two domains of the transcriptional activator. This proximity causes transcription of a reporter gene which is operably linked to a transcriptional regulatory site responsive to the transcriptional activator, and expression of the reporter gene can be detected and used to score for the interaction of the bait and fish proteins.

In accordance with the present invention, the method includes providing a host cell, preferably a yeast cell, e.g., Kluyverei lactis, Schizosaccharomyces pombe, Ustilago maydis, Saccharomyces cerevisiae, Neurospora crassa, Aspergillus niger, Aspergillus nidulans, Pichia pastoris, Candida tropicalis, and Hansenula polymorpha, though most preferably S cerevisiae or S. pombe. The host cell contains a reporter gene having a binding site for the DNA-binding domain of a transcriptional activator used in the bait protein, such that the reporter gene expresses a detectable gene product when the gene is transcriptionally activated. The first chimeric gene may be present in a chromosome of the host cell, or as part of an expression vector.

The host cell also contains a first chimeric gene which is capable of being expressed in the host cell. The gene encodes a chimeric protein, e.g., the “bait protein” which comprises (i) a DNA-binding domain that recognizes the responsive element on the reporter gene in the host cell, and (ii) bait protein, such as a cell- or tissue-specific F-box polypeptide or substrate polypeptide sequence.

A second chimeric gene is also provided which is capable of being expressed in the host cell, and encodes the “fish fusion protein.” In one embodiment, both the first and the second chimeric genes are introduced into the host cell in the form of plasmids. Preferably, however, the first chimeric gene is present in a chromosome of the host cell and the second chimeric gene is introduced into the host cell as part of a plasmid.

Preferably, the DNA-binding domain of the first hybrid protein and the transcriptional activation domain of the second hybrid protein are derived from transcriptional activators having separable DNA-binding and transcriptional activation domains. For instance, these separate DNA-binding and transcriptional activation domains are known to be found in the yeast GAL4 protein, and are known to be found in the yeast GCN4 and ADR1 proteins. Many other proteins involved in transcription also have separable binding and transcriptional activation domains which make them useful for the present invention, and include, for example, the LexA and VP16 proteins. It will be understood that other (substantially) transcriptionally-inert DNA-binding domains may be used in the subject constructs; such as domains of ACE1, λcI, lac repressor, jun or fos. In another embodiment, the DNA-binding domain and the transcriptional activation domain may be from different proteins. The use of a LexA DNA binding domain provides certain advantages. For example, in yeast, the LexA moiety contains no activation function and has no known effect on transcription of yeast genes. In addition, use of LexA allows control over the sensitivity of the assay to the level of interaction (see, for example, the Brent et al. PCT publication WO94/10300).

In preferred embodiments, any enzymatic activity associated with the bait or fish proteins is inactivated, e.g., dominant negative mutants of an SCF complex component and the like can be used or mutant substrate polypeptides lacking ubiquitin-accepting lysine residues.

Continuing with the illustrated example, the cell- or tissue-specific F-box protein/substrate-mediated interaction, if any, between the bait and fish fusion proteins in the host cell, therefore, causes the activation domain to activate transcription of the reporter gene. The method is carried out by introducing the first chimeric gene and the second chimeric gene into the host cell, and subjecting that cell to conditions under which the bait and fish fusion proteins and are expressed in sufficient quantity for the reporter gene to be activated. The formation of an cell- or tissue-specific F-box protein/substrate complex results in a detectable signal produced by the expression of the reporter gene. Accordingly, the formation of a complex in the presence of a test compound to the level of cell- or tissue-specific F-box protein/substrate complex in the absence of the test compound can be evaluated by detecting the level of expression of the reporter gene in each case.

In an illustrative embodiment, Saccharomyces cerevisiae YPB2 cells are transformed simultaneously with a plasmid encoding a GAL4 db-SIP fusion and with a plasmid encoding the GAL4ad domain fused in-frame to a coding sequence for a p27 polypeptide. Moreover, the strain is transformed such that the GAL4-responsive promoter drives expression of a phenotypic marker. For example, the ability to grow in the absence of histidine can depend on the expression of the LacZ gene. When the LacZ gene is placed under the control of a GAL4-responsive promoter, the yeast cell will turn blue in the presence of β-gal if a functional GAL4 activator has been reconstituted through the interaction of a cell- or tissue-specific F-box protein and a substrate. Thus, a convenient readout method is provided. Other reporter constructs will be apparent, and include, for example, reporter genes which produce such detectable signals as selected from the group consisting of an enzymatic signal, a fluorescent signal, a phosphorescent signal and drug resistance.

A similar method modifies the interaction trap system by providing a “relay gene” which is regulated by the transcriptional complex formed by the interacting bait and fish proteins. The gene product of the relay gene, in turn, regulates expression of a reporter gene, the expression of the latter being what is scored in the modified ITS assay. Fundamentally, the relay gene can be seen as a signal inverter.

As set out above, in the standard ITS, interaction of the fish and bait fusion proteins results in expression of a reporter gene. However, where inhibitors of the interaction are sought, a positive readout from the reporter gene nevertheless requires detecting inhibition (or lack of expression) of the reporter gene.

In the inverted ITS system, the fish and bait proteins positively regulate expression of the relay gene. The relay gene product is in turn a repressor of expression of the reporter gene. Inhibition of expression of the relay gene product by inhibiting the interaction of the fish and bait proteins results in concomitant relief of the inhibition of the reporter gene, e.g., the reporter gene is expressed. For example, the relay gene can be the repressor gene under control of a promoter sensitive to the cell- or tissue-specific F-box protein/substrate complex described above. The reporter gene can accordingly be a positive signal, such as providing for growth (e.g., drug selection or auxotrophic relief), and is under the control of a promoter which is constitutively active, but can be suppressed by the repressor protein. In the absence of an agent which inhibits the interaction of the fish and bait protein, the repressor protein is expressed. In turn, that protein represses expression of the reporter gene. However, an agent which disrupts binding of the cell- or tissue-specific F-box polypeptide and substrate proteins results in a decrease in repressor expression, and consequently an increase in expression of the reporter gene as repression is relieved. Hence, the signal is inverted.

In other embodiments, the invention provides assays, such as derived in formats set forth above, which identify agents capable of disrupting the interaction between p19^{skp1}, p45^{skp2}, Rbx1 or a cullins, or a substrate protein and atrophin-1, or another cell- or tissue-specific F-box protein, e.g., such as the competitive binding assays described above.

One aspect of the present invention provides reconstituted protein preparations, e.g., purified protein combinations, including a cell- or tissue-specific F-box polypeptide plus one or more of the following proteins (or polypeptides or fusion proteins derived therefrom): an E1, an E2, a substrate protein, ubiquitin, a cullins, Rbx1, p19^{skp1} and/or p45^{skp2}.

In still further embodiments of the present assay, the ubiquitin-conjugating system is generated in whole cells, taking advantage of cell culture techniques to support the subject assay. For example, as described below, the ubiquitin-conjugating system (including the substrate polypeptide and detection means) can be constituted in a eukaryotic cell culture system, including mammalian and yeast cells. Advantages to generating the subject assay in an intact cell include the ability to detect inhibitors which are functional in an environment more closely approximating that which therapeutic use of the inhibitor would require, including the ability of the agent to gain entry into the cell. Furthermore, certain of the in vivo embodiments of the assay, such as examples given below, are amenable to high through-put analysis of candidate agents.

The components of the ubiquitin-conjugating system, including the substrate polypeptide and cell- or tissue-specific F-box polypeptides, can be endogenous to the cell selected to support the assay. Alternatively, some or all of the components can be derived from exogenous sources. For instance, fusion proteins can be introduced into the cell by recombinant techniques (such as through the use of an expression vector), as well as by microinjecting the fusion protein itself or mRNA encoding the fusion protein.

In any case, the cell is ultimately manipulated after incubation with a candidate inhibitor in order to facilitate detection of ubiquitination or ubiquitin-mediated degradation of the substrate polypeptide. As described above for assays performed in reconstituted protein mixtures or lysate, the effectiveness of a candidate inhibitor can be assessed by measuring direct characteristics of the substrate polypeptide, such as shifts in molecular weight by electrophoretic means or detection in a binding assay. For these embodiments, the cell will typically be lysed at the end of incubation with the candidate agent, and the lysate manipulated in a detection step in much the same manner as might be the reconstituted protein mixture or lysate, e.g., described above.

Indirect measurement of ubiquitination of the substrate polypeptide can also be accomplished by detecting a biological activity associated with the substrate polypeptide that is either attenuated by ubiquitin-conjugation or destroyed along with the substrate polypeptide by ubiquitin-dependent proteolytic processes. As set out above, the use of fusion proteins comprising the substrate polypeptide and an enzymatic activity are representative embodiments of the subject assay in which the detection means relies on indirect measurement of ubiquitination of the substrate polypeptide by quantitating an associated enzymatic activity.

In other embodiments, the biological activity of the substrate polypeptide can be assessed by a monitoring changes in the phenotype of the targeted cell. For example, the detection means can include a reporter gene construct which includes a transcriptional regulatory element that is dependent in some form on the level of the substrate protein. The substrate protein can be provided as a fusion protein with a domain which binds to a DNA element of the reporter gene construct. The added domain of the fusion protein can be one which, through its DNA-binding ability, increases or decreases transcription of the reporter gene. Which ever the case may be, its presence in the fusion protein renders it destructible by a ubiquitin-mediated pathway. Accordingly, the level of expression of the reporter gene will vary with the stability of the fusion protein.

The reporter gene product may be a detectable label, such as luciferase or β-galactosidase, and may be produced in the intact cell. The label can be measured in a subsequent lysate of the cell. However, the lysis step is preferably avoided, and providing a step of lysing the cell to measure the label will typically only be employed where detection of the label cannot be accomplished in whole cells.

Moreover, in the whole cell embodiments of the subject assay, the reporter gene construct can provide, upon expression, a selectable marker. A reporter gene includes any gene that expresses a detectable gene product, which may be RNA or protein. Preferred reporter genes are those that are readily detectable. The reporter gene may also be included in the construct in the form of a fusion gene with a gene that includes desired transcriptional regulatory sequences or exhibits other desirable properties. For instance, the product of the reporter gene can be an enzyme which confers resistance to antibiotic or other drug, or an enzyme which complements a deficiency in the host cell (i.e. thymidine kinase or dihydrofolate reductase). To illustrate, the aminoglycoside phosphotransferase encoded by the bacterial transposon gene Tn5 neo can be placed under transcriptional control of a promoter element responsive to the level of target substrate polypeptide present in the cell. Such embodiments of the subject assay are particularly amenable to high through-put analysis in that proliferation of the cell can provide a simple measure of inhibition of the ubiquitin-mediated degradation of the substrate polypeptide.

Other examples of reporter genes include, but are not limited to CAT (chloramphenicol acetyl transferase) (Alton and Vapnek (1979), Nature 282: 864-869) luciferase, and other enzyme detection systems, such as beta-galactosidase; firefly luciferase (deWet et al. (1987), Mol. Cell. Biol. 7:725-737); bacterial luciferase (Engebrecht and Silverman (1984), PNAS 1: 4154-4158; Baldwin et al. (1984), Biochemistry 23: 3663-3667); alkaline phosphatase (Toh et al. (1989) Eur. J. Biochem. 182: 231-238, Hall et al. (1983) J. Mol. Appl. Gen. 2: 101), human placental secreted alkaline phosphatase (Cullen and Malim (1992) Methods in Enzymol. 216:362-368).

The amount of transcription from the reporter gene may be measured using any method known to those of skill in the art to be suitable. For example, specific mRNA expression may be detected using Northern blots or specific protein product may be identified by a characteristic stain or an intrinsic activity.

In preferred embodiments, the product of the reporter gene is detected by an intrinsic activity associated with that product. For instance, the reporter gene may encode a gene product that, by enzymatic activity, gives rise to a detection signal based on color, fluorescence, or luminescence.

The amount of expression from the reporter gene is then compared to the amount of expression in either the same cell in the absence of the test compound or it may be compared with the amount of transcription in a substantially identical cell that lacks a component of the Ub-pathway, such as a cell- or tissue-specific F-box protein activity, etc.

The present invention also makes available yeast cells which contain an F-box protein null mutation. As described herein, these strains can be complemented using human genes, and thus “humanized” yeast strains can be created for in vivo drug screen, e.g., which comprise a human cell- or tissue-specific F-box protein homolog and (optionally) a human substrate protein. The strain can be further manipulated to be “humanized” with respect to other biochemical steps in the F-box protein-mediated ubiquitination of a substrate protein. For example, conditional inactivation of the relevant yeast UBC enzyme with concomitant expression of the human UBC homolog, or alternatively, replacement of other yeast genes involved in ubiquitination with their human homologs, provides a humanized system whereby the substrate protein can be ubiquitinated by a mechanism which approximates the F-box protein-dependent ubiquitination that occurs in vertebrate cells.

In still another embodiment, the difference between the human cell- or tissue-specific F-box proteins and a yeast F-box protein can be exploited, e.g., by the use of differential screening techniques, to identify antifungal agents which have a specificity for the yeast SCF complex relative to the mammalian SCF complex. Thus, lead compounds which act specifically on pathogens, such as fungus involved in mycotic infections, can be developed. By way of illustration, any of the above assay formats, generated to compare inhibition of a fungal F-box protein with a mammalian cell- or tissue-specific F-box protein, can be used to screen for agents which may ultimately be useful for inhibiting at least one fungus implicated in such mycosis as candidiasis, aspergillosis, mucormycosis, blastomycosis, geotrichosis, cryptococcosis, chromoblastomycosis, coccidioidomycosis, conidiosporosis, histoplasmosis, maduromycosis, rhinosporidosis, nocaidiosis, para-actinomycosis, penicilliosis, monoliasis, or sporotrichosis. For example, if the mycotic infection to which treatment is desired is candidiasis, the subject assays can comprise comparing the relative effectiveness of a test compound at inhibiting the activity of a mammalian cell- or tissue-specific F-box protein with its effectiveness towards inhibiting the activity of an F-box gene cloned from yeast selected from the group consisting of Candida albicans, Candida stellatoidea, Candida tropicalis, Candida parapsilosis, Candida krusei, Candida pseudotropicalis, Candida quillermondii, or Candida rugosa. Likewise, the present assay can be used to identify anti-fungal agents which may have therapeutic value in the treatment of aspergillosis by making use of the subject assays derived from F-box protein genes cloned from yeast such as Aspergillus fumigatus, Aspergillus flavus, Aspergillus niger, Aspergillus nidulans, or Aspergillus terreus. Where the mycotic infection is mucormycosis, the F-box protein can be derived from yeast such as Rhizopus arrhizus, Rhizopus oryzae, Absidia corymbifera, Absidia ramosa, or Mucor pusillus. Sources of other yeast F-box proteins for comparison with a mammalian cell- or tissue-specific F-box protein include the pathogen Pneumocystis carinii. Exemplary F-box protein genes from human pathogens and other lower eukaryotes are provided by, for example, GenBank Accession numbers: X96763 (Candida albican) and X05625 (Saccharomyces cerevisiae).

11. Microarray Analysis of Gene Expression

Many different methods are known in the art for measuring gene expression. Classical methods include quantitative RT-PCR, Northern blots and ribonuclease protection assays. Such methods may be used to examine expression of individual genes as well as entire gene clusters. However, as the number of genes to be examined increases, the time and expense may become prohibitive.

Large scale detection methods allow faster, less expensive analysis of the expression levels of many genes simultaneously. Such methods typically involve an ordered array of probes affixed to a solid substrate. Each probe is capable of hybridizing to a different set of nucleic acids. In one method, probes are generated by amplifying or synthesizing a substantial portion of the coding regions of various genes of interest. These genes are then spotted onto a solid support. mRNA samples are obtained, converted to cDNA, amplified and labeled (usually with a fluorescence label). The labeled cDNAs are then applied to the array, and cDNAs hybridize to their respective probes in a manner that is linearly related to their concentration. Detection of the label allows measurement of the amount of each cDNA adhered to the array.

Many methods for performing such DNA array experiments are well known in the art. Exemplary methods are described below but are not intended to be limiting.

Arrays are often divided into microarrays and macroarrays, where microarrays have a much higher density of individual probe species per area. Microarrays may have as many as 1000 or more different probes in a 1 cm² area. There is no concrete cut-off to demarcate the difference between micro- and macroarrays, and both types of arrays are contemplated for use with the invention. However, because of their small size, microarrays provide great advantages in speed, automation and cost-effectiveness.

Microarrays are known in the art and consist of a surface to which probes that correspond in sequence to gene products (e.g., cDNAs, mRNAs, oligonucleotides) are bound at known positions. In one embodiment, the microarray is an array (i.e., a matrix) in which each position represents a discrete binding site for a product encoded by a gene (e.g., a protein or RNA), and in which binding sites are present for products of most or almost all of the genes in the organism's genome. In a preferred embodiment, the “binding site” (hereinafter, “site”) is a nucleic acid or nucleic acid analogue to which a particular cognate cDNA can specifically hybridize. The nucleic acid or analogue of the binding site can be, e.g., a synthetic oligomer, a full-length cDNA, a less-than full length cDNA, or a gene fragment.

Although in a preferred embodiment the microarray contains binding sites for products of all or almost all genes in the target organism's genome, such comprehensiveness is not necessarily required. Usually the microarray will have binding sites corresponding to at least 100 genes and more preferably, 500, 1000, 4000 or more. The most preferred arrays will have about 98-100% of the genes of a particular organism represented. Preferably, the microarray has binding sites for genes relevant to testing and confirming a biological network model of interest. Several exemplary human microarrays are publicly available. The Affymetrix GeneChip HUM 6.8K is an oligonucleotide array composed of 7,070 genes. A microarray with 8,150 human cDNAs was developed and published by Research Genetics (Bittner et al., 2000, Nature 406:443-546).

The probes to be affixed to the arrays are typically polynucleotides. These DNAs can be obtained by, e.g., polymerase chain reaction (PCR) amplification of gene segments from genomic DNA, cDNA (e.g., by RT-PCR), or cloned sequences. PCR primers are chosen, based on the known sequence of the genes or cDNA, that result in amplification of unique fragments (i.e. fragments that do not share more than 10 bases of contiguous identical sequence with any other fragment on the microarray). Computer programs are useful in the design of primers with the required specificity and optimal amplification properties. See, e.g., Oligo pl version 5.0 (National Biosciences). In the case of binding sites corresponding to very long genes, it will sometimes be desirable to amplify segments near the 3′ end of the gene so that when oligo-dT primed cDNA probes are hybridized to the microarray, less-than-full length probes will bind efficiently. Random oligo-dT priming may also be used to obtain cDNAs corresponding to as yet unknown genes, known as ESTs. Certain arrays use many small oligonucleotides corresponding to overlapping portions of genes. Such oligonucleotides may be chemically synthesized by a variety of well known methods. Synthetic sequences are between about 15 and about 500 bases in length, more typically between about 20 and about 50 bases. In some embodiments, synthetic nucleic acids include non-natural bases, e.g., inosine. As noted above, nucleic acid analogues may be used as binding sites for hybridization. An example of a suitable nucleic acid analogue is peptide nucleic acid (see, e.g., Eghohn et al., 1993, PNA hybridizes to complementary oligonucleotides obeying the Watson-Crick hydrogen-bonding rules, Nature 365:566-568; see also U.S. Pat. No. 5,539,083).

In an alternative embodiment, the binding (hybridization) sites are made from plasmid or phage clones of genes, cDNAs (e.g., expressed sequence tags), or inserts therefrom (Nguyen et al., 1995, Differential gene expression in the murine thymus assayed by quantitative hybridization of arrayed cDNA clones, Genomics 29:207-209). In yet another embodiment, the polynucleotide of the binding sites is RNA.

The nucleic acids or analogues are attached to a solid support, which may be made from glass, plastic (e.g., polypropylene, nylon), polyacrylamide, nitrocellulose, or other materials. A preferred method for attaching the nucleic acids to a surface is by printing on glass plates, as is described generally by Schena et al., 1995, Science 270:467-470. This method is especially useful for preparing microarrays of cDNA. (See also DeRisi et al., 1996, Nature Genetics 14:457-460; Shalon et al., 1996, Genome Res. 6:639-645; and Schena et al., 1995, Proc. Natl. Acad. Sci. USA 93:10539-11286). Each of the aforementioned articles is incorporated by reference in its entirety for all purposes.

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Science 251:767-773; Pease et al., 1994, Proc. Natl. Acad. Sci. USA 91:5022-5026; Lockhart et al., 1996, Nature Biotech 14:1675; U.S. Pat. Nos. 5,578,832; 5,556,752; and 5,510,270, each of which is incorporated by reference in its entirety for all purposes) or other methods for rapid synthesis and deposition of defined oligonucleotides (Blanchard et al., 1996, 11: 687-90). When these methods are used, oligonucleotides of known sequence are synthesized directly on a surface such as a derivatized glass slide. Usually, the array produced is redundant, with several oligonucleotide molecules per RNA. Oligonucleotide probes can be chosen to detect alternatively spliced mRNAs.

Other methods for making microarrays, e.g., by masking (Maskos and Southern, 1992, Nuc. Acids Res. 20:1679-1684), may also be used. In principal, any type of array, for example, dot blots on a nylon hybridization membrane (see Sambrook et al., Molecular Cloning—A Laboratory Manual (2nd Ed.), Vol. 1-3, Cold Spring Harbor Laboratory, Cold Spring Harbor, N.Y., 1989, which is incorporated in its entirety for all purposes), could be used, although, as will be recognized by those of skill in the art, very small arrays will be preferred because hybridization volumes will be smaller.

The nucleic acids to be contacted with the microarray may be prepared in a variety of ways. Methods for preparing total and poly(A)+ RNA are well known and are described generally in Sambrook et al., supra. Labeled cDNA is prepared from mRNA by oligo dT-primed or random-primed reverse transcription, both of which are well known in the art (see e.g., Klug and Berger, 1987, Methods Enzymol. 152:316-325). Reverse transcription may be carried out in the presence of a dNTP conjugated to a detectable label, most preferably a fluorescently labeled dNTP. Alternatively, isolated mRNA can be converted to labeled antisense RNA synthesized by in vitro transcription of double-stranded cDNA in the presence of labeled dNTPs (Lockhart et al., 1996, Nature Biotech. 14:1675). The cDNAs or RNAs can be synthesized in the absence of detectable label and may be labeled subsequently, e.g., by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent.

When fluorescent labels are used, many suitable fluorophores are known, including fluorescein, lissamine, phycoerythrin, rhodamine (Perkin Elmer Cetus), Cy2, Cy3, Cy3.5, Cy5, Cy5.5, Cy7, Fluor X (Amersham) and others (see, e.g., Kricka, 1992, Academic Press San Diego, Calif.).

In another embodiment, a label other than a fluorescent label is used. For example, a radioactive label, or a pair of radioactive labels with distinct emission spectra, can be used (see Zhao et al., 1995, Gene 156:207; Pietu et al., 1996, Genome Res. 6:492). However, use of radioisotopes is a less-preferred embodiment.

Nucleic acid hybridization and wash conditions are chosen so that the population of labeled nucleic acids will specifically hybridize to appropriate, complementary nucleic acids affixed to the matrix. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches).

Optimal hybridization conditions will depend on the length (e.g., oligomer versus polynucleotide greater than 200 bases) and type (e.g., RNA, DNA, PNA) of labeled nucleic acids and immobilized polynucleotide or oligonucleotide. General parameters for specific (i.e., stringent) hybridization conditions for nucleic acids are described in Sambrook et al., supra, and in Ausubel et al., 1987, Current Protocols in Molecular Biology, Greene Publishing and Wiley-Interscience, New York, which is incorporated in its entirety for all purposes. Non-specific binding of the labeled nucleic acids to the array can be decreased by treating the array with a large quantity of non-specific DNA—a so-called “blocking” step.

When fluorescently labeled probes are used, the fluorescence emissions at each site of a transcript array can be, preferably, detected by scanning confocal laser microscopy. When two fluorophores are used, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al., 1996, Genome Research 6:639-645). In a preferred embodiment, the arrays are scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., 1996, Genome Res. 6:639-645 and in other references cited herein. Alternatively, the fiber-optic bundle described by Ferguson et al., 1996, Nature Biotech. 14:1681-1684, may be used to monitor mRNA abundance levels at a large number of sites simultaneously. Fluorescent microarray scanners are commercially available from Affymetrix, Packard BioChip Technologies, BioRobotics and many other suppliers.

Signals are recorded, quantitated and analyzed using a variety of computer software. In one embodiment the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores is preferably calculated. The ratio is independent of the absolute expression level of the cognate gene, but is useful for genes whose expression is significantly modulated by drug administration, gene deletion, or any other tested event.

According to the method of the invention, the relative abundance of an mRNA in two cells or cell lines is scored as a perturbation and its magnitude determined (i.e., the abundance is different in the two sources of mRNA tested), or as not perturbed (i.e., the relative abundance is the same). As used herein, a difference between the two sources of RNA of at least a factor of about 25% (RNA from one source is 25% more abundant in one source than the other source), more usually about 50%, even more often by a factor of about 2 (twice as abundant), 3 (three times as abundant) or 5 (five times as abundant) is scored as a perturbation. Present detection methods allow reliable detection of difference of an order of about 2-fold to about 5-fold, but more sensitive methods are expected to be developed.

Preferably, in addition to identifying a perturbation as positive or negative, it is advantageous to determine the magnitude of the perturbation. This can be carried out, as noted above, by calculating the ratio of the emission of the two fluorophores used for differential labeling, or by analogous methods that will be readily apparent to those of skill in the art.

In one embodiment of the invention, transcript arrays reflecting the transcriptional state of a cell of interest are made by hybridizing a mixture of two differently labeled sets of cDNAs, to the microarray. One cell is a cell of interest, while the other is used as a standardizing control. The relative hybridization of each cell's cDNA to the microarray then reflects the relative expression of each gene in the two cell. For example, to assess gene expression in a variety of breast cancers, Perou et al. (2000, supra) hybridized fluorescently-labeled cDNA from each tumor to a microarray in conjunction with a standard mix of cDNAs obtained from a set of breast cancer cell lines. In this way, each tumor is compared against the same standard, and may readily be compared against each other.

In preferred embodiments, the data obtained from such experiments reflects the relative expression of each gene represented in the microarray. Expression levels in different samples and conditions may now be compared using a variety of statistical methods.

In an exemplary embodiment, cDNA libraries obtained from different cell- or tissue-types are differentially displayed on a microarray to identify genes which are specifically expressed in a particular cell or tissue type. The cDNA libraries may be enriched for F-box containing sequences prior to differential display by any one of a variety of art recognized methods. For example, the cDNA library may be amplified using a primer which binds to an F-box specific sequence. Alternatively, a cDNA library may be enriched for F-box containing sequences by isolating sequences based on their ability to hybridize to an oligonucleotide capable of binding to F-box specific sequences. Preferably such primers or oligonucleotides are capable of binding to the F-box consensus sequence as defined herein. Such primer and oligonucleotides may be degenerate (i.e. a mixture of sequences) so as to bind to a variety of F-box sequences.

In another embodiment, cDNA libraries obtained from cells harvested from diseased vs. normal animals are differentially displayed on a microarray to identify genes whose expression increases or decreases in the diseased state as compared to the normal state. For example, cells from tissues of interest may be harvested from a diseased animal and a normal animal and cDNA libraries may be produced and differentially displayed on a microarray system to compare gene expression patterns from cells in the diseased animal as compared to cells from the normal animal. In a specific example, muscle wasting may be induced in a mouse by subjecting the mouse to fasting conditions. Muscle cells may then be harvested from the fasted mouse and cDNA libraries obtained may be profiled and compared to the profile of a cDNA library obtained from the muscle cells of a non-fasted mouse. Increased expression of a gene in the muscles of the fasted mouse as compared to the normal mouse may indicate genes which are involved in muscle wasting, e.g., genes encoding proteins involved in protein degradation, etc.

Exemplification

The invention now being generally described, it will be more readily understood by reference to the following examples which are included merely for purposes of illustration of certain aspects and embodiments of the present invention, and are not intended to limit the invention.

### EXAMPLE 1

### Identification of a Muscle Specific F-Box Protein, Atrophin-1

In several different animal models of atrophy, muscles exhibit a common series of adaptations that indicate an activation of the Ub-proteasome pathway. For example, these muscles have an increased content of Ub-protein conjugates (the critical intermediates in this pathway) and of mRNA encoding Ub, certain ubiquitination enzymes (e.g., E2₁₄ₖ and E3α) and multiple proteasome subunits. During these studies, several genes have been identified (e.g. genes for Ub, E2₁₄ₖ), whose expression rises in all of these models of muscle wasting, despite the general fall in mRNA content when muscles atrophy.

To truly understand the mechanisms of muscle wasting, it would be of great value to obtain a comprehensive picture of the spectrum of genes whose transcription rises or falls in different types of atrophy. Therefore, we recently began to use the new microarray transcription profiling, or “chip” technology (from Incyte). Using this system, we measured mRNA levels of approximately 10,000 different genes derived from human and mouse libraries, and identified those genes whose expression was significantly altered in atrophying muscle. Our initial chip analysis utilized muscles from fasted mice, because food deprivation had been shown previously to lead to increased proteolysis through stimulation of the ubiquitin-proteasome pathway. We also chose this experimental model because the molecular events in the muscle appear to mimic those in other human diseases (e.g. diabetes, cancer cachexia, sepsis).

Our microarray analysis demonstrated that most mRNAs in the atrophying muscles did not change significantly in amount, and many were found to be decreased by 1.8 to 3-fold. However, while a number of anticipated components were increased (i.e., poly Ub, multiple proteasome subunits), several unidentified gene fragments (ESTs) on our microarrays also increased 2 to 9-fold. We have named the gene corresponding to the EST which increased by the greatest proportion (9-fold) in muscle atrophy due to fasting, atrophin-1.

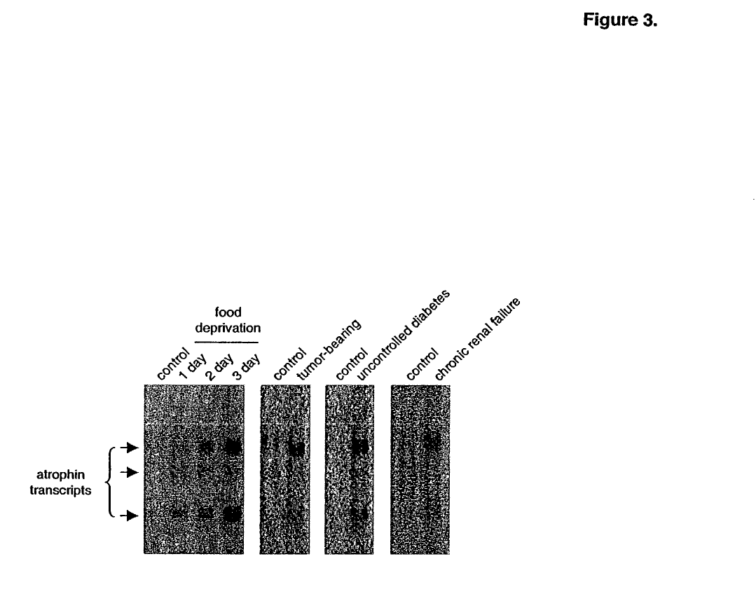

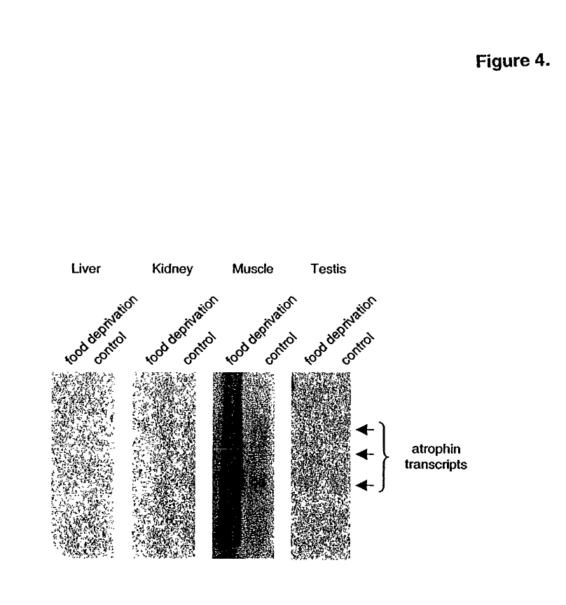

We have also measured the tissue distribution of this gene, as well as its expression in muscles from other catabolic animals. We found a marked increase in atrophin-1 expression in atrophying muscles from rats bearing Yoshida ascities hepatoma, after streptozotocin administration (a model of acute, uncontrolled diabetes), and after 5/6 nephrectomy to induce uremia (chronic renal failure) (FIG. 3). Indeed, its expression appears to be activated even more than that of the polyub gene making it, as far as we know, the mRNA most responsive to catabolic states. Furthermore, we failed to find significant expression of atrophin-1 in other mouse tissues including liver, kidney and testis (FIG. 4).

Finally, we isolated the full-length sequence of atrophin-1 from a mouse cDNA library we generated from the muscle of fasted mice (FIG. 5). The gene encodes a 40 kD protein that contains an F-box near its C-terminus (FIG. 5). Proteins containing F-boxes make up a growing family of polypeptides that are the substrate recognition components of SCF Ub-protein ligases (E3s) (FIG. 2). It is likely that atrophin-1 makes up part of an SCF-type E3 that is specific to muscle and plays an important role in muscle atrophy. Atrophin-1 probably recognizes critical substrate(s) in muscle that are ubiquitinated and degraded. These substrates might be regulatory components of the muscle cell or parts of the myofibrillar apparatus.

All of the above-cited references and publications are hereby incorporated by reference.

### Equivalents

Those skilled in the art will recognize, or be able to ascertain using no more than routine experimentation, many equivalents to the specific embodiments of the invention described herein. Such equivalents are intended to be encompassed by the following claims.

We claim:

1. An isolated and/or recombinant cell- or tissue-specific F-box protein.

2. The isolated and/or recombinant cell- or tissue-specific F-box protein of claim 1, which is expressed specifically in muscle tissue.

3. The isolated and/or recombinant cell- or tissue-specific F-box protein of claim 2, which protein comprises an amino acid sequence identical or homologous to the amino acid sequence shown in FIG. 5B.

4. The isolated and/or recombinant cell- or tissue-specific F-box protein of claim 1, wherein expression of the F-box protein is at least 2-fold higher in one cell- or tissue-type as compared to another cell- or tissue-type.

5. The isolated and/or recombinant cell- or tissue-specific F-box protein of claim 2, which protein comprises an amino acid sequence encoded by a nucleic acid which hybridizes under stringent conditions to the nucleotide sequence set forth in FIG. 5A.

6. The isolated and/or recombinant cell- or tissue-specific F-box protein of claim 1, which protein has an endogenous ubiquitin ligase activity and/or is a component of a ubiquitin ligase.

7. The isolated and/or recombinant cell- or tissue-specific F-box protein of claim 6, wherein the polypeptide mediates ubiquitination of a cell-type specific or tissue-type specific substrate protein.

8. The isolated and/or recombinant cell- or tissue-specific F-box protein of claim 1, which polypeptide is capable of interacting with at least one other protein selected from the group consisting of ubiquitin, a component of ubiquitin ligase, a skp1 protein, a cullins protein, an Rbx1 protein and a ubiquitin conjugating enzyme.

9. The isolated and/or recombinant cell- or tissue-specific F-box protein of claim 1, which polypeptide is at least 75% homologous to the amino acid sequence shown in FIG. 5B.

10. The isolated and/or recombinant cell- or tissue-specific F-box protein of claim 1, which polypeptide is a vertebrate F-box protein.

11. The isolated and/or recombinant cell- or tissue-specific F-box protein of claim 10, which polypeptide is a mammalian F-box protein.

12. The isolated and/or recombinant cell- or tissue-specific F-box protein of claim 11, which polypeptide comprises the amino acid sequence set forth in FIG. 5B.

13. An isolated nucleic acid comprising a nucleotide sequence encoding a cell- or tissue-specific F-box polypeptide, or a nucleotide sequence complementary thereto, said F-box polypeptide including an amino acid sequence identical or homologous to the amino acid sequence set forth in FIG. 5B, or a portion thereof.

14. The nucleic acid of claim 13, which nucleic acid hybridizes under stringent conditions to a nucleic acid probe comprising a nucleotide sequence represented by at least 60 consecutive nucleotides of the sequence shown in FIG. 5A, or a sequence complementary thereto.

15. The nucleic acid of claim 14, which nucleic acid comprises the nucleotide sequence set forth in FIG. 5A.

16. An isolated nucleic acid comprising a nucleotide sequence encoding a vertebrate cell- or tissue-specific F-box polypeptide.

17. The nucleic acid of claim 13 or 16, further comprising a transcriptional regulatory sequence operably linked to said nucleotide sequence so as to render said nucleic acid suitable for use as an expression vector.

18. An expression vector, capable of replicating in at least one of a prokaryotic cell and eukaryotic cell, comprising the expression vector of claim 17.

19. A host cell transfected with the expression vector of claim 18 and expressing said recombinant polypeptide.

20. A method of producing a recombinant cell- or tissue-specific F-box polypeptide comprising culturing the cell of claim 19 in a cell culture medium to express said recombinant polypeptide and isolating said recombinant polypeptide from said cell culture.

21. A transgenic animal having cells which harbor a transgene comprising the nucleic acid of claim 13 or 16, or in which a gene comprising said nucleic acid is disrupted.

22. An isolated nucleic acid which selectively hybridizes under high stringency conditions to at least ten nucleotides of the sequence set forth in FIG. 5A or complementary sequences thereof, which nucleic acid can specifically detect or amplify a nucleic acid sequence of a vertebrate cell- or tissue-specific F-box gene.

23. The nucleic acid of claim 22, which is nucleic acid is labeled.

24. A reconstituted protein mixture comprising a cell- or tissue-specific F-box polypeptide and a substrate protein.

25. The reconstituted protein mixture of claim 24, wherein the cell- or tissue-specific F-box polypeptide is atrophin-1.

26. The reconstituted protein mixture of claim 24, wherein the substrate protein is a regulatory component of a muscle cell or a component of the myofibrillar apparatus.

27. An assay for identifying an inhibitor of cell- or tissue-specific F-box protein-mediated ubiquitination, comprising:
(i) providing a ubiquitin-conjugating system including a substrate polypeptide, an SCF complex including one or more cell- or tissue-specific F-box polypeptides and ubiquitin, under conditions which promote ubiquitination of the substrate polypeptide by the SCF complex;
(ii) contacting the ubiquitin-conjugating system with a candidate agent;
(iii) measuring a level of ubiquitination of the substrate polypeptide in the presence of the candidate agent; and
(iv) comparing the measured level of ubiquitination in the presence of the candidate agent with ubiquitination of the substrate polypeptide in the absence of the candidate agent,
wherein a statistically significant decrease in ubiquitination of the substrate polypeptide in the presence of the candidate agent is indicative of an inhibitor of cell- or tissue-specific F-box protein-mediated ubiquitination.

28. The assay of claim 27, wherein the ubiquitin-conjugating system comprises a reconstituted protein mixture.

29. The assay of claim 27, wherein the ubiquitin-conjugating system comprises a cell lysate.

30. The assay of claim 27, wherein the SCF complex includes a polypeptide comprising the sequence set forth in FIG. 5B.

31. The assay of claim 27, wherein the substrate protein is a regulatory component of a muscle cell or a component of the myofibrillar apparatus.

32. The assay of claim 27, wherein the ubiquitin is provided in a form selected from a group consisting of:
(i) an unconjugated ubiquitin, in which case the ubiquitin-conjugating system further comprises an E1 ubiquitin-activating enzyme (E1), an E2 ubiquitin-conjugating enzyme (E2), and adenosine triphosphate;
(ii) an activated E1:ubiquitin complex, in which case the ubiquitin-conjugating system further comprises an E2;
(iii) an activated E2:ubiquitin complex; and
(iv) an activated E3:ubiquitin complex.

33. The assay of claim 27, wherein the ubiquitin-conjugating system further comprises an E2 ubiquitin conjugating enzyme.

34. The assay of claim 27, wherein at least one of the ubiquitin and the substrate polypeptide comprises a detectable label, and the level of ubiquitination of the substrate polypeptide is quantified by detecting the label in at least one of the substrate polypeptide, the ubiquitin, and ubiquitin-conjugated substrate polypeptide.

35. The assay of claim 34, wherein the label group is selected from a group consisting of radioisotopes, fluorescent compounds, enzymes, and enzyme co-factors.

36. The assay of claim 34, wherein the detectable label comprises a polypeptide having a measurable activity, and the substrate polypeptide is fusion protein including the detectable label.

37. The assay of claim 27, wherein the amount of ubiquitination of the substrate polypeptide is quantified by an immunoassay.

38. The assay of claim 27, wherein the amount of ubiquitination of the substrate polypeptide is quantified by chromatography or electrophoresis.

39. The assay of claim 27, wherein the ubiquitin-conjugating system comprises a host cell expressing the substrate polypeptide and the SCF complex.

40. The assay of claim 39, wherein the host cell expresses a recombinant cell- or tissue-specific F-box polypeptide.

41. The assay of claim 27, wherein the ubiquitin-conjugating system further comprises a cullins polypeptide, a Skp1 polypeptide and/or an Rbx1 polypeptide.

42. An assay for identifying an inhibitor of an interaction between a substrate polypeptide and an SCF complex including a cell- or tissue-specific F-box protein, comprising:
(i) providing a reaction system including the substrate polypeptide and an SCF complex including a cell- or tissue-specific F-box protein, wherein the substrate polypeptide and the SCF complex interact;
(ii) contacting the reaction system with a candidate agent;
(iii) measuring formation of complexes containing the substrate polypeptide and the SCF complex in the presence of the candidate agent; and
(iv) comparing the measured formation of complexes in the presence of the candidate agent with complexes formed in the absence of the candidate agent,
wherein a statistically significant decrease in the formation of complexes in the presence of the candidate agent is indicative of an inhibitor of the interaction of the substrate polypeptide and the SCF complex.

43. The assay of claim 42, wherein the cell- or tissue-specific F-box protein is atrohpin-1.

44. The assay of claim 42, wherein the substrate is a regulatory component of a muscle cell or a component of the myofibrillar apparatus.

45. The assay of claim 42, wherein the reaction system comprises a reconstituted protein mixture.

46. The assay of claim 42, wherein the reaction system comprises a cell lysate.

47. The assay of claim 42, wherein the reaction system comprises a cell.

48. The assay of claim 42, wherein the substrate polypeptide and the cell- or tissue-specific F-box protein are provided as fusion proteins in an interaction trap system.

49. The assay of claim 27, which comprises a further step of preparing a pharmaceutical preparation of one or more compounds identified as inhibitors of cell- or tissue-specific F-box protein-mediated ubiquitination.

50. A method for diagnosing a muscle wasting disorder in a patient, comprising:
(i) ascertaining the level of expression of an F-box polypeptide comprising the sequence set forth in FIG. 5B in a sample of muscle cells from the patient; and
(ii) diagnosing the presence or absence of a muscle wasting disorder utilizing, at least in part, the ascertained level of expression or activity of the F-box polypeptide;
wherein an increased level of expression of the F-box polypeptide or F-box polypeptide-dependent ubiquitination activity in the sample, relative to a control sample of non-muscle cells, correlates with the presence of a muscle wasting disorder.

51. The method of claim 50, wherein the patient is suffering from chachexia, cachexia secondary to infection or malignancy, cachexia secondary to human acquired immune deficiency syndrome AIDS, rheumatoid arthritis, rheumatoid spondylitis, osteoarthritis, gouty arthritis, sepsis, septic shock, endotoxic shock, gram negative sepsis, toxic shock syndrome, adult respiratory distress syndrome, cerebral malaria, chronic pulmonary inflammatory disease, silicosis, pulmonary sarcoidosis, bone resorption diseases, reperfusion injury, graft vs. host reaction, allograft rejections, Crohn's disease, ulcerative colitis, or pyresis, multiple sclerosis, autoimmune diabetes, systemic lupus erythematosis, prolonged inactivity or prolonged exposure to a microgravity environment.

52. The method of claim 50, further comprising the step of applying a treatment to the patient which inhibits the expression and/or activity of the F-box polypeptide.

53. The method of claim 52, wherein the muscle wasting disorder is associated with chachexia, cachexia secondary to infection or malignancy, cachexia secondary to human acquired immune deficiency syndrome AIDS, rheumatoid arthritis, rheumatoid spondylitis, osteoarthritis, gouty arthritis, sepsis, septic shock, endotoxic shock, gram negative sepsis, toxic shock syndrome, adult respiratory distress syndrome, cerebral malaria, chronic pulmonary inflammatory disease, silicosis, pulmonary sarcoidosis, bone resorption diseases, reperfusion injury, graft vs. host reaction, allograft rejections, Crohn's disease, ulcerative colitis, or pyresis, multiple sclerosis, autoimmune diabetes, systemic lupus erythematosis, prolonged inactivity or prolonged exposure to a microgravity environment.

54. A method for treating a pateint suffering from a muscle wasting disorder, comprising administering to the pateint an amount of an atrohpin-1 inhibitor effective to inhibit the expression and/or activity of atrophin-1.

55. The method of claim 54, wherein the muscle wasting disorder is associated with chachexia, cachexia secondary to infection or malignancy, cachexia secondary to human acquired immune deficiency syndrome AIDS, rheumatoid arthritis, rheumatoid spondylitis, osteoarthritis, gouty arthritis, sepsis, septic shock, endotoxic shock, gram negative sepsis, toxic shock syndrome, adult respiratory distress syndrome, cerebral malaria, chronic pulmonary inflammatory disease, silicosis, pulmonary sarcoidosis, bone resorption diseases, reperfusion injury, graft vs. host reaction, allograft rejections, Crohn's disease, ulcerative colitis, or pyresis, multiple sclerosis, autoimmune diabetes, systemic lupus erythematosis, prolonged inactivity or prolonged exposure to a microgravity environment.

54. A method for maintaining or increasing the muscle mass of an animal, comprising administering to the animal an amount of an atrohpin-1 inhibitor effective to inhibit the expression and/or activity of atrophin-1.

55. The method of claim 54, wherein the animal is a human.

56. The method of claim 54, wherein the animal is a livestock animal.

57. The method of claim 56, wherein the animal is a cow, pig, goat or sheep.

58. A method for stimulating the proliferation of muscle stem cells, comprising contacting the stem cells with a compound capable of inhibiting the expression and/or activity of atrophin-1.

59. A transgenic livestock animal which is disrupted for atrophin-1.

60. A method for inhibiting protein degredation in muscle tissue of a patient without substanitally affecting protein degradation in other tissues, compising administering to the patient an amount of an atrophin-1 inhibitor effective to inhibit the expression and/or activity of atrophin-1.
